SEQUENCE LISTING (9 sequences)

SEQ ID NO: 1 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 2 - protein, 132 aa, Homo sapiens
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

SEQ ID NO: 3 - protein, 524 aa, Human papillomavirus
MSVGDSYPNRLFIVDVLCPFVKPHLTPPLFYIVLIHFHFDTFVFFLYLLRFNKRAIMSIRAKRRKRASPTDLYRTCKQAGTCPPDIIPRVEQNTLADKILKWGSLGVFFGGLGIGTGSGTGGRTGYIPVGSRPTTVVDIGPTPRPPVVIEPVGASEPSIVTLVEDSSIINAGASHPTFTGTGGFEVTTSTVTDPAVLDITPSGTSVQVSSSSFLNPLYTEPAIVEAPQTGEVSGHVLVSTATSGSHGYEEIPMQTFATSGGSGTEPISSTPLPGVRRVAGPRLYSRANQQVQVRDPAFLARPADLVTFDNPVYDPQKTIIFQHPDLHEPPDPDFLDIVALHRPALTSRRGTVRFSRLGRRATLRTRSGKQIGARVHFYHDISPIGTEELEMEPLLPPASTDNTDMLYDVYADSDVLQPLLDELPAAPRGSLSLADTAVSATSASTLRGSTTVPLSSGIDVPVYTGPDIEPPNVPGMGPLIPVAPSLPSSVYIFGGDYYLMPSYVLWPKRRKRVHYFFADGFVAA

SEQ ID NO: 4 - protein, 13 aa, Human papillomavirus
LVEETSFIDAGAP

SEQ ID NO: 5 - DNA, 21 bp, Artificial Sequence
  Description of Artificial Sequence Synthetic oligonucleotide
gcaagucccuguacuauuatt

SEQ ID NO: 6 - DNA, 23 bp, Artificial Sequence
  Description of Artificial Sequence Synthetic oligonucleotide
cgggaugcuuugaacauugaatt

SEQ ID NO: 7 - DNA, 21 bp, Artificial Sequence
  Description of Artificial Sequence Synthetic oligonucleotide
uaauaguacagggacuugctt

SEQ ID NO: 8 - DNA, 23 bp, Artificial Sequence
  Description of Artificial Sequence Synthetic oligonucleotide
uucaauguucaaagcaucccgtt

SEQ ID NO: 9 - protein, 6 aa, Artificial Sequence
  Description of Artificial Sequence Synthetic 6xHis tag
HHHHHH

(19) United States
(12) Patent Application Publication
(10) Pub. No.: US 2011/0086044 A1
(43) Pub. Date: Apr. 14, 2011

(54) Compositions and Methods to Inhibit HPV Infection

(75) Inventor: W. Martin Kast, Los Angeles, CA (US)

(73) Assignee: University of Southern California

(21) Appl. No.: 12/892,859

(22) Filed: Sep. 28, 2010

Related U.S. Application Data
(60) Provisional application No. 61/246,884, filed on Sep. 29, 2009.

(51) Int. Cl. A61K 39/395; C12N 5/00; A61K 38/02; A61K 31/7105; C12Q 1/70

(52) U.S. Cl. 424/172.1; 435/375; 514/3.7; 514/44 A; 435/5

(57) ABSTRACT

This invention provides compositions and method for inhibiting and treating an HPV infection of LC or tissue containing LC by administering to the LC or tissue an effective amount of an agent that inhibits HPV binding to annexin A2 (ANXA2) present on the surface of the cell, thereby inhibiting HPV infection. It also provides methods to design antiviral and/or anticancer agents for cancers that harbor HPV.

### CROSS REFERENCE TO RELATED APPLICATION

This application claims the benefit under 35 U.S.C. §119(e) of U.S. Provisional Ser. No. 61/246,884, filed Sep. 29, 2009, the content of which is incorporated by reference in its entirety.

### STATEMENT OF GOVERNMENT SUPPORT

This invention was made with government support under Grant No. RO1 CA74397 awarded by the National Institutes of Health. The U.S. Government has certain rights in the invention.

### BACKGROUND

Throughout this disclosure technical and patent literature are identified by a bibliographic citation or by an Arabic number within parenthesis, the full bibliographic citation for which can be found at the end of this disclosure immediately preceding the claims. The contents of these disclosures as well as those identified with this application are hereby incorporated by reference in their entirety to more fully describe the state of the art to which this disclosure pertains.

Human papillomavirus (HPV) are a family of DNA viruses and the high-risk HPV types are associated with the development of anogenital cancers. In the United States, an estimated 75% of the sexually active population acquires at least one genital HPV type during their lifetime. The risk of acquiring HIV is increased by co-infection with other sexually transmitted diseases (STDs), possibly due to shared risk factors. HIV-1-infected women have a higher prevalence, incidence, and persistence of HPV infection (3). Concurrent infection with multiple HPV types is more common in HPV/HIV-1-coinfected women and is associated with an increased risk for cervical, vulvar and vaginal cancers. As a result, HIV-1-infected women are 5-fold more likely to develop cervical intraepithelial neoplasia (CIN), the precursor to cervical cancer, than HIV-negative women. HIV-positive men are also victims of HPV-related cancers. Anal cancer is becoming one of the most common AIDS-related cancers in men who have sex with men, and penile and oral cancer incidence rates are on the rise. Therefore, a broad spectrum therapeutic for HPV infection and/or persistence would be able to benefit both women and men living with HIV/AIDS who are at high risk for developing HPV-associated cancers.

Human papillomavirus (HPV) causes an increased incidence of several different types of cancer in HIV-infected individuals because of their immune suppression. With increased life expectancies due to advances in AIDS therapies and with high incidence of HPV co-infection, there is an urgent need to develop therapeutic strategies to reduce the risk and prevent the development of HPV-associated malignancies. Persistent high risk human papillomavirus (HPV) infection also is a significant cause of anogenital cancers in HIV-positive individuals. Thus, a need exists to prevent and inhibit HPV infectivity in at-risk individuals. This disclosure satisfies this need and provides related advantages as well.

### SUMMARY OF THE INVENTION

Secretory leukocyte protease inhibitor (SLPI) is a serine protease inhibitor found in mucosal fluids of the genital tract and has been shown to inhibit infection of macrophages by HIV by blocking the interaction of HIV with annexin A2 (ANXA2). One of the mechanisms by which HPV escapes immunity is inducing tolerance via antigen presentation in the absence of co-stimulation by Langerhans cells (LC), the antigen-presenting cells at the site of HPV and HIV infection. Applicants have confirmed that HPV infection is mediated in part through interaction with ANXA2 and have determined that secretory leukocyte protease inhibitor (SLPI) can block uptake of HPV and HIV by LC. In a further aspect, the HPV binds to ANXA2 by binding to the ANXA2/p11 heterotetramer complex that can be on either or both of ANXA1 and/or p11.

This disclosure provides a method for inhibiting HPV infection of LC or tissue containing LC comprising, or alternatively consisting essentially of, or yet further consisting of, administering to the LC or tissue an effective amount of an agent that inhibits HPV binding to annexin A2 (ANXA2) present on the surface of the cell, thereby inhibiting HPV infection. In a further aspect, the HPV binds to ANXA2 by binding to the ANXA2/p11 heterotetramer complex that can be on either or both of ANXA1 and/or p11. In one aspect, the agent is SLPI. In another aspect, the agent is an agent that upregulates the expression of SLPI in the tissue. In another aspect, the agent is administered in combination with a TLR agonist. The second agent can be co-administered or administered prior to or subsequent to administration of the agent that inhibits HPV infection.

In another aspect, this disclosure provides a method for treating or preventing HPV infection in a subject having or at risk of HPV infection, comprising, or alternatively consisting essentially of, or yet further consisting of, administering to the subject an effective amount of an agent that inhibits HPV binding to ANXA2, thereby preventing or treating HPV infection in the subject. In a further aspect, the HPV binds to ANXA2 by binding to the ANXA2/p11 heterotetramer complex that can be on either or both of ANXA1 and/or p11. In one aspect, the agent is SLPI. In another aspect, the agent is an agent that upregulates the expression of SLPI in the subject. In another aspect, the agent is administered in combination with a TLR agonist. The second agent can be co-administered or administered prior to or subsequent to administration of the agent that inhibits HPV infection.

In another aspect, this disclosure provides a method for preventing or inhibiting HPV-related pathologies in a subject having or at risk of an HPV infection, comprising, or alternatively consisting essentially of, or yet further consisting of, administering to the subject an effective amount of an agent that inhibits HPV binding to ANXA2, thereby preventing or treating HPV-related pathologies in the subject. In a further aspect, the HPV binds to ANXA2 by binding to the ANXA2/p11 heterotetramer complex that can be on either or both of ANXA1 and/or p11. Non-limiting examples of such pathologies are cervical, vulvar and vaginal cancers, cervical intraepithelial neoplasia (CIN), anal cancer, penile cancer, and oral cancer. In one aspect, the agent is SLPI. In another aspect, the agent is an agent that upregulates the expression of SLPI in the subject. In another aspect, the agent is administered in combination with a TLR agonist. The second agent can be co-administered or administered prior to or subsequent to administration of the agent that prevents or inhibits HPV infection.

In yet another aspect, this disclosure provides a composition comprising, or alternatively consisting essentially of, or yet further consisting of, an effective amount of an agent that inhibits or prevents HPV binding to ANXA2 and a pharmaceutically acceptable carrier. In a further aspect, the HPV binds to ANXA2 by binding to the ANXA2/p11 heterotetramer complex that can be on either or both of ANXA1 and/or p11. The composition can further comprise, or alternatively consist essentially of, or yet further consist of, an effective amount of a second agent such as a TLR agonist.

In yet another aspect, this disclosure provides a method to determine if a test agent is suitable for inhibiting or preventing HPV infection of a LC or tissue containing LC or alternatively or in addition, testing for antivirals or anticancer agents that utilize the HPV receptor for entry, the method comprising, or alternatively consisting essentially of, or yet further consisting of, (a) administering to a first tissue sample an amount of the test agent; (b) administering to a second tissue sample an effective amount of an agent that inhibits binding of HPV to ANXA2; and (c) comparing the binding, uptake, and gene expression of HPV in the first tissue sample to the binding, uptake, and gene expression in the second tissue sample and/or third tissue sample, wherein the test agent is suitable for inhibiting or preventing HPV infection if the HPV viral titer of the first tissue sample is similar to the second tissue sample. In a further aspect, the HPV binds to ANXA2 by binding to the ANXA2/p11 heterotetramer complex that can be on either or both of ANXA1 and/or p11.

Further provided is use of the above-mentioned compositions in the manufacture of a medicament for inhibiting HPV infection in a LC, tissue containing LC or a subject having or at risk of HPV infection. The medicaments may further comprise additional pharmaceuticals or agents that induce a localized immune response. These may be combined with pharmaceutically acceptable carriers that are suitable for the modes of administration.

In yet another aspect, this disclosure provides a kit for preventing or inhibiting HPV infection in a LC or tissue containing LC or a subject at risk of or having an HPV infection, comprising, or alternatively consisting essentially of, or yet further consisting of, an effective amount of an agent that inhibits HPV binding to ANXA2 in a pharmaceutically acceptable carrier and instructions for use in preventing or inhibiting HPV infection. In one aspect, the agent is SLPI. In another aspect, the agent is an agent that upregulates the expression of SLPI in the subject. In another aspect, the agent is administered in combination with a TLR agonist. The second agent can be co-administered or administered prior to or subsequent to administration of the agent that prevents or inhibits HPV infection.

Agents that inhibit binding of HPV to ANXA are also provided, including but not limited to, a secretory leukocyte protease inhibitor (SLPI), an agent that enhances expression of SLPI, a peptide comprising the extracellular domain of an ANXA2 receptor, an siRNA directed at ANXA2, a polynucleotide encoding the siRNA, a peptide comprising an HPVL2 region or an antibody that recognizes and binds ANXA2. In a further aspect, the HPV binds to ANXA2 by binding to the ANXA2/p11 heterotetramer complex that can be on either or both of ANXA1 and/or p11.

### BRIEF DESCRIPTION OF THE FIGURES

FIG. 1 shows results the ANXA2 and 6×His tag immunoblot analysis of LC eluates isolated from a peptide pull-down assay. LC were incubated with either no peptide or 50 μg/5×10⁵ cells of 6×His-L2₁₀₈₋₁₂₀ peptide and subsequently cross-linked with DTSSP. Cells were then lysed and mixed with a Ni-NTA agarose slurry overnight and ten fractions were eluted. Non-reduced eluates were electrophoresed, transferred to nitrocellulose and probed with an anti-ANXA2 antibody or an anti-6×His antibody, followed by a peroxidase-labeled secondary antibody. Antibody binding was detected with enhanced chemiluminescence.

FIG. 2 is a chart showing SLPI blocks HPV16 L1L2 VLP uptake into LC, but not HPV16 L1 VLP. LC were left untreated or incubated with 30 μg human recombinant SLPI (R&D Systems) for 1 h at 4° C. Subsequently, the cells were washed and incubated with CFDA-SE-labeled HPV16 L1 VLP or HPV16 L1L2 VLP at 37° C. After 15 min, cells were washed at 4° C., then fixed with 1% paraformaldehyde and analyzed for intracellular CFDA-SE by flow cytometry. Percent of VLP uptake was calculated using the following equation: [(MFI experimental plus SLPI)−(MFI background)/[(MFI experimental untreated cells)−(MFI background)]×100. Shown is the average uptake of 3 experiments (±SEM). *p=0.04 compared to untreated LC.

FIG. 3 shows exemplary amino acid sequences of human annexin 2 isoform 2 (SEQ ID NO: 1); human SPLI (SEQ ID NO: 2); and HPV L2 polypeptide (SEQ ID NO: 3).

FIG. 4 shows that HPV16L1 VLP and HPV16L1L2 VLP enter and travel through LC in different cellular compartments. HPV16L1 VLP and HPV16L1L2 VLP were labeled with different fluorescent dyes and incubated with LC for various periods of time. At each given time point, the cells were visualized with confocal microscopy. One representative experiment of three is shown.

FIG. 5 shows that HPV16 L2₁₀₈₋₁₂₀ peptide inhibits binding of HPV16L1L2 VLP to LC. LC were incubated with increasing concentrations of the L2₁₀₈₋₁₂₀ peptide and subsequently incubated with HPV16L1L2 VLP. HPV16L1L2 VLP remaining on the surface of LC were detected using a L1 specific conformational antibody (H16.V5). Binding was assessed by flow cytometry. These data are expressed as the mean of three separate experiments±SD (*P<0.05 as determined by a two-tailed, unpaired t-test, as compared to untreated LC).

FIGS. 6A & B show that The HPV16 L2₁₀₈₋₁₂₀ peptide pulls down the ANXA2 heterotetramer. A. LC were incubated with either no peptide or (6×)His-L2₁₀₈₋₁₂₀ peptide and subsequently cross-linked with DTSSP. Cells were lysed and mixed with a Ni-NTA agarose slurry and eluted. Reduced eluate 5 was electrophoresed and silver stained. The unique band right above ˜39 kDa was isolated and analyzed by mass spectrometry. One representative experiment of two is shown. B. LC were incubated with either no peptide or (6×)His-L2₁₀₈₋₁₂₀ peptide and subsequently cross-linked with DTSSP. Cells were then lysed and mixed with a Ni-NTA agarose slurry overnight and eluted. Eluates were then electrophoresed, transferred to nitrocellulose and probed with either an anti-ANXA2 or an anti-ANXA2 light chain antibody. ANXA2 is also known as p11. One representative experiment of two is shown.

FIGS. 7A & B are charts showing that SLPI inhibits the uptake of HPV16L1L2 VLP by LC. A. LC were incubated with increasing concentrations of SLPI, then incubated with CFDA-SE labeled HPV16L1L2 VLP for 15 min. Uptake of CFDA-SE labeled HPV16L1L2 VLP by LC was assessed by flow cytometry. The mean percentage of uptake ±SEM of three separate experiments is presented (*P<0.05 by a two-tailed, paired t-test, as compared to the negative control). B. LC were incubated with SLPI (30 ug/ml) and exposed to CFDA-SE labeled HPV16L1 VLP. Uptake of CFDA-SE labeled HPV16 VLP by LC was assessed by flow cytometry. The mean percentage uptake±SEM of three separate experiments is presented.

FIGS. 8A & B are gel pictures showing downregulation of ANXA2 inhibits uptake of HPV16L1L2 VLP. A. LC were transfected using the Amaxa Nucleofector kit without siRNA (Untreated), with control siRNA or with two different ANXA2 siRNA sequences. The cells were incubated for 6 days before analysis of ANXA2 protein expression by immunoblot. GAPDH served as the loading control. One representative experiment of three is shown. B. LC were transfected using the Amaxa Nucleofector kit without siRNA (Untreated), with control siRNA or with ANXA2 siRNA #2. The cells were incubated for 6 days and exposed to CFDA-SE labeled HPV16L1L2 VLP for 15 min. Uptake was assessed by flow cytometry. These data are expressed as the mean of four separate experiments ±SD (*P<0.05 as determined by a two-tailed, paired t-test, as compared to untreated LC).

FIG. 9 illustrates a unifying theory of HPV uptake. HPV binds to the host cell surface through HSPG, CyPB, α₆β_{1/4} integrins and/or tetraspanins (CD63, CD151), either singularly or in complex. The binding of HPV to the cell surface sets off several signaling cascades that lead to alterations in host cell function and facilitate virion internalization. The binding of integrins by HPV recruits integrin-associated proteins such as talin and activates FAK. Talin activates PIP5K, which synthesizes the second messenger PI(4,5)P₂ locally. This local accumulation of PI(4,5)P₂ has been shown to recruit ANXA2 heterotetramers to the membrane. The activation of FAK by integrin binding leads to the activation of src-family kinases (SFK). Activated SFK phosphorylate Tyr²³ on ANXA2 heterotetramers, causing them to translocate to the cell surface. This proposed mechanism allows for ANXA2 heterotetramers to appear focally at the cell surface at the site of HPV binding. After HPV interacts with the host cell primary receptor(s), it undergoes a conformational change and subsequent furin cleavage of the L2 protein. This conformational change leads to a decrease in affinity for the primary receptor(s) and a potential increase in affinity for the ANXA2 heterotetramer. Upon binding to the ANXA2 heterotetramer, HPV is internalized through a clathrin-, caveolin-, dynamin-, flotillin- and lipid-raft independent endosomal pathway. As the endosome transitions into a late endosome/lysosome, decreasing pH leads to viral uncoating.

### DETAILED DESCRIPTION OF THE INVENTION

### Definitions

The practice of the present disclosure will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); Current Protocols in Immunology (J. E. Coligan, et. al. eds., (1997)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

The terms “inhibit” or “antagonize” is intended to mean an decrease of amount or activity of the target. In one aspect, they refer to decrease of the infection and replication of HPV in a cell or tissue in vitro and/or in vivo.

An “agonist”, as used herein, refers to a drug or other chemical that can bind a receptor on a cell to produce a physiologic reaction typical of a naturally occurring substance. The efficacy of an agonist may be positive, causing an increase in the receptor's activity.

“Administration”, as used herein, refers to the delivery of a medication, such as the agent of the disclosure, which inhibits HPV infection, to an appropriate location of the subject, where a therapeutic effect is achieved. Non-limiting examples include oral dosing, intracutaneous injection, direct application to target area proximal areas on the skin, or applied on a patch. Various physical and/or mechanical technologies are available to permit the sustained or immediate topical or transdermal administration of macromolecules (such as, peptides).

“Topical administration” refers to delivery of a medication by application to the mucosal membrane or skin. Non-limiting examples of topical administration include any methods described under the definition of “administration” pertaining to delivery of a medication to appropriate area.

A penetration or permeation enhancer refers to a chemical composition or mechanical/electrical device that can increase the transdermal drug delivery efficiency. In one aspect, a penetration or permeation enhancer is soluble in the formulation and act to reduce the barrier properties of human skin. The list of potential skin permeation enhancers is long, but can be broken down into three general categories: lipid disrupting agents (LDAs), solubility enhancers, and surfactants. LDAs are typically fatty acid-like molecules proposed to fluidize lipids in the human skin membrane. Solubility enhancers act by increasing the maximum concentration of drug in the formulation, thus creating a larger concentration gradient for diffusion. Surfactants are amphiphilic molecules capable of interacting with the polar and lipid groups in the skin (see e.g. Francoeur et al. (1990) Pharm. Res. 7:621-7; U.S. Pat. No. 5,503,843).

A “composition” is intended to mean a combination of active agent, cell or population of cells and another compound or composition, inert (for example, a detectable agent or label or biocompatible scaffold) or active, such as a growth and/or differentiation factor.

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

For topical use, the pharmaceutically acceptable carrier is suitable for manufacture of creams, ointments, jellies, gels, solutions, suspensions, etc. Such carriers are conventional in the art, e.g., for topical administration with polyethylene glycol (PEG) or carboxymethylcellulose. These formulations may optionally comprise additional pharmaceutically acceptable ingredients such as diluents, stabilizers, and/or adjuvants.

A “subject” of diagnosis or treatment is a cell, tissue, or a mammal, including a human. Non-human animals subject to diagnosis or treatment include, for example, murine, such as rats, mice, canine, such as dogs, leporids, such as rabbits, livestock, sport animals, and pets. In some embodiments, the “subject” is a HPV-infected patient who may have developed peripheral tolerance towards HPV or a HIV/HPV-infected patients who is slightly more immune compromised.

An “effective amount” is an amount sufficient to effect beneficial or desired results. An effective amount can be administered in one or more administrations, applications or dosages.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Alternatively, a positive control is an agent exhibiting a desired biological response and a negative control is one that is known not to exhibit the desired biological response.

As used herein, the terms “treating,” “treatment” and the like are used herein to mean obtaining a desired pharmacologic and/or physiologic effect. The effect can be prophylactic in terms of completely or partially preventing an infection or disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed or at risk of an infection or a disorder, but has not yet been diagnosed as having it; inhibiting a disorder, i.e., arresting its development; and/or relieving or ameliorating the symptoms of disorder, e.g., HPV infection or cervical cancer. As is understood by those skilled in the art, “treatment” can include systemic amelioration of the symptoms associated with the pathology and/or a delay in onset of symptoms.

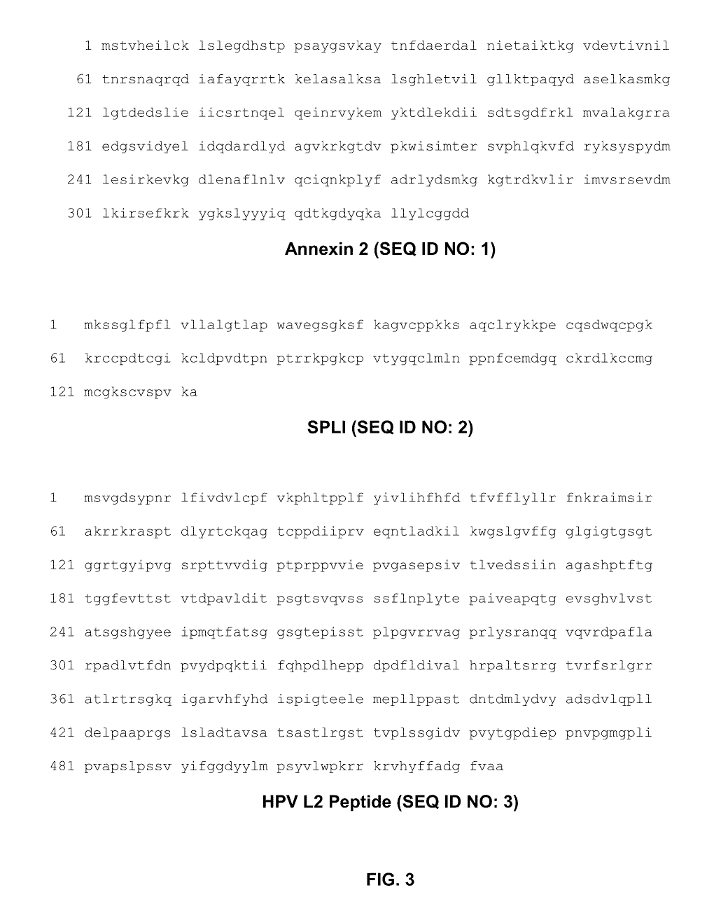

Annexin is a family of proteins, including ANXA2. The amino acid sequence of which is known for various species in the art. A non-limiting exemplary sequence is published at GenBank under Accession No. NP_{—}004030 (Homo sapiens) and Entrez Gene: 302 and UniProt: P07355 (last accessed on Sep. 28, 2009). An example of a reported sequence is provided in FIG. 3.

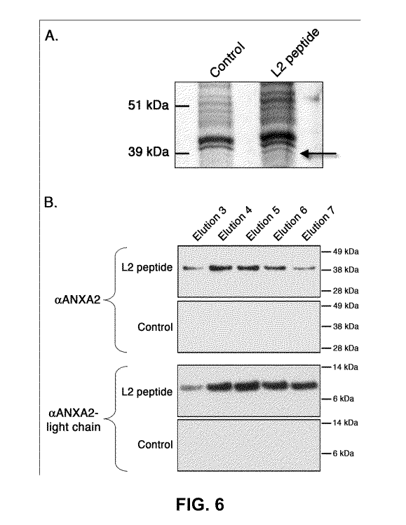

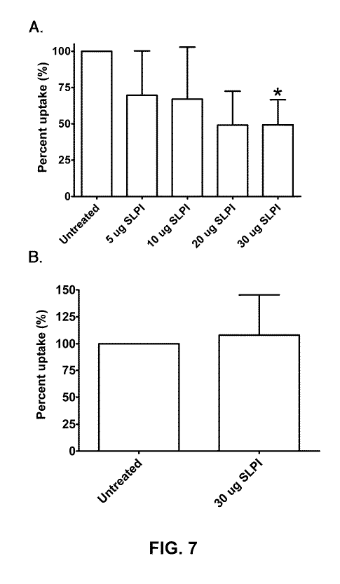

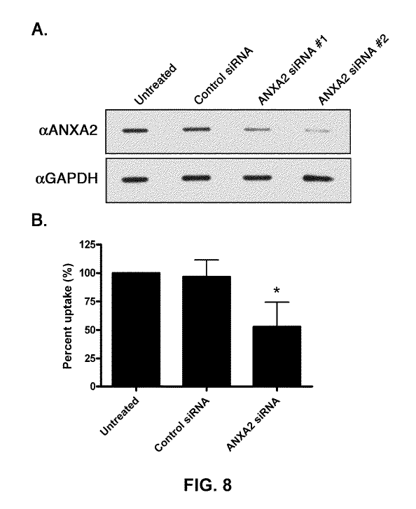

As used herein, and unless specifically stated otherwise, when referring to binding to ANXA2, e.g., HPV L2 binding to ANXA2, the term intends binding to ANXA2 and any other complex comprising ANXA2. For example, ANXA2 can exist as a heterotetramer with p11. Without being bound by theory, when HPVL2 binds ANXA2, it could be binding to an ANXA2/p11 complex which comprises ANXA2 but is not the direct binding partner of HPVL2. See for example FIG. 6 showing that the L2 peptide pulled down ANXA2 and p11.

Secretory leukocyte protease inhibitor (SLPI) is a potent inhibitor of human leukocyte elastase (EC 3.4.21.37) and cathepsin G (EC 3.4.21.20) and of human trypsin (EC 3.4.21.4) has been purified from human parotid secretions. The complete amino acid sequence of this protein has been determined and is reported in Thompson and Ohlsson (1986) PNAS 83(18):6692-6696. The authors report that the sequence suggests that the protein has two domains of about 54 amino acids, each of which contains four disulfide bonds. On the basis of a limited homology to other protease inhibitors, the antielastase and antitrypsin activities are thought to be properties of the C-terminal and N-terminal domains, respectively. As used herein, the term SLPI intends proteins or polypeptides having this or other sequences reported to be SLPI, as reported for example in GenBank Accession No. CAA28187 (last accessed on Sep. 28, 2009), the sequence of which is reproduce in FIG. 3. and those having at least 75%, or alternatively at least 80%, or alternatively at least 85%, or alternatively at least 90%, or alternatively at least 95%, or alternatively at least 97% sequence identity to this and other reported sequences.

### Toll Like Receptors and Agonists

Among mammals, there are 11 Toll like receptors (TLRs) expressed by LC. LCs express several like TLR 1, 2, 3, 5, 6 and 10 (Flacher et al. (2006) J. Immunol. 177:7959-7967).

In some embodiments, the TLR agonist used in the disclosure is one or more of the above recited 11 TLR agonists.

In some embodiments, the TLR agonist used in the disclosure is one or more of the TLR 3, TLR 7, TLR 8, TLR 9, or a combination thereof. In some embodiments, the TLR agonist used in the disclosure is one or more of the TLR 3, TLR 8, TLR 9, or a combination thereof. In some embodiments, the TLR agonist used in the disclosure is one or more of the TLR 8, TLR 9, or a combination thereof. In some embodiments, the TLR agonist used in the disclosure is one or more of the TLR 3, TLR 8, or a combination thereof. In some embodiments, the TLR agonist used in the disclosure is one or more of the TLR 3, TLR 9, or a combination thereof. In some embodiments, the TLR agonist used in the disclosure is TLR 3. In some embodiments, the TLR agonist used in the disclosure is TLR 8. In some embodiments, the TLR agonist used in the disclosure is TLR 9.

In some embodiments, the TLR agonist is a single stranded RNA, double stranded RNA, or a synthetic small molecule.

Examples of TLR 3 agonist include, but are not limited to, polyinosine-polycytidylic acid (poly I:C), a synthetic analog of dsRNA; poly-ICLC; and poly-ICR.

Poly-ICLC drug is a synthetic complex of carboxymethylcellulose, polyinosinic-polycytidylic acid, and poly-L-lysine double-stranded RNA. There are at least four interrelated clinical actions of poly-ICLC, any of which (alone or in combination) might be responsible for its anti-tumor and anti-viral activity. These are 1) its induction of interferons; 2) its broad immune enhancing effect; 3) its activation of specific enzymes, especially oligoadenylate synthetase (OAS) and the p68 protein kinase (PKR); and 4) its broad gene regulatory actions.

Another example of TLR3 agonist is poly-ICR (Poly IC-Poly Arginine), which may have greater biologic effects at much lower concentrations. Poly-ICR is a TLR3 agonist that when combined with a disease-specific antigen can induce both cytotoxic (T-cell) and antibody (B-cell) immune responses against that antigen. Cytotoxic T-cells, also referred to as CD8 T-cells, are required to target and eliminate pathogen-infected or cancerous cells. Antibodies or B-cells, are required to protect against an infection caused by a pathogen. Poly-ICR, therefore, has potential utility in both the therapeutic and prophylactic areas of immunotherapy and vaccine development. This novel and potent immunomodulator works with the immune system to induce dendritic cell maturation, along with a broad range of inflammatory cytokines and chemokines, to facilitate the prevention and treatment of infectious diseases or cancer.

Small molecule examples of TLR 7 agonist include, but are not limited to, CL264 (Adenine analog); Gardiquimod™ (imidazoquinoline compound); Imiquimod (imidazoquinoline compound); and Loxoribine (guanosine analogue).

Examples of TLR 8 agonist include, but are not limited to, single-stranded RNAs and E. coli RNA.

In some embodiments, the TLR agonist activates dual TLR receptors such as, but not limited to, TLR 7/8 agonist. Examples of TLR 7/8 agonist include, but are not limited to, CL075 (thiazoloquinoline compound); CL097 (water-soluble R848, imidazoquinoline compound); poly(dT) (thymidine homopolymer phosphorothioate ODN); and R848 (imidazoquinoline compound).

CL075 (3M002, structure shown below) is a thiazoloquinolone derivative that stimulates TLR8 in human PBMC.

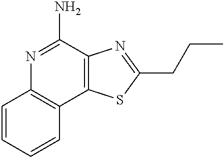

It activates NF-κB and triggers preferentially the production of TNF-α and IL-12. CL075 may also induce the secretion of IFN-α through TLR7 but to a lesser extend. It can induce the activation of NF-κB at 0.4 μM (0.1 μg/ml) in TLR8-transfected HEK293 cells, and ˜10 times more CL075 to activate NF-κB in TLR7-transfected HEK293 cells.

CL097 (structure shown below) is a highly water-soluble derivative of the imidazoquinoline compound R848 (≦20 mg/ml).

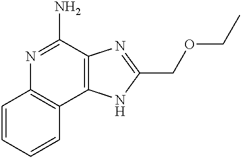

Similarly to R848, CL097 is a TLR7 and TLR8 ligand. It can induce the activation of NF-κB at 0.4 μM (0.1 μg/ml) in TLR7-transfected HEK293 cells and at 4 μM (1 μg/ml) in TLR8-transfected HEK293 cells.

Poly(dT), a thymidine homopolymer phosphorothioate ODN, is a modulator of human TLR7 and TLR8. In combination with an imidazoquinoline, such as R848 and CL075, it increases TLR8-mediated signaling but abolishes TLR7-mediated signaling. A co-incubation of poly(dT) and an imidazoquinoline can induce NF-κB activation in HEK293 cells transfected with murine TLR8— and primary TLR8-expressing mouse cells.

R848 (structure shown below) is an imidazoquinoline compound with potent anti-viral activity.

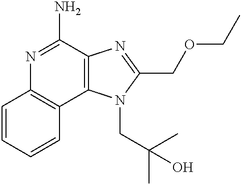

This low molecular weight synthetic molecule activates immune cells via the TLR7/TLR8 MyD88-dependent signaling pathway. R848 has been shown to trigger NE-κB activation in cells expressing murine TLR8 when combined with poly(dT) (Gorden et al. (2006) J. Immunol. 177: 6584-6587).

Toll-like receptor 9 (TLR9) is activated by specific unmethylated CpG-containing sequences in bacterial DNA or synthetic oligonucleotides (ODNs) in the endosomal compartment. These specific sequences called CpG motifs are present at high frequency in bacterial DNA but rare in mammalian DNA. The methylation status is a distinction between bacterial and mammalian DNA. Unmethylated ODNs including a CpG motif can mimic the effects of bacterial DNA, inducing B-cell proliferation and activating cells of the myeloid lineage.

Examples of TLR 9 agonist include, but are not limited to, stimulatory ODNs such as, CpG ODNs, Control ODNs, and Labeled ODNs; and E. coli DNA such as, E. coli DNA of and E. coli ssDNA.

Stimulatory CpG ODNs can be of three types, types A, B and C, which differ in their immune-stimulatory activities. They induce differentially the stimulation of human and murine immune cells in vitro, a species-specificity that is also observed with non-responsive cells such as HEK293 cells transfected with human or mouse TLR9. Type A CpG ODNs are characterized by a phosphodiester central CpG-containing palindromic motif and a phosphorothioate poly-G string. They induce high IFN-α production from plasmacytoid dendritic cells (pDC) but are weak stimulators of TLR9-dependent NF-kappaB signaling. Type B CpG ODNs contain a full phosphorothioate backbone with one or more CpG dinucleotides. They strongly activate B cells but stimulate weakly IFN-α secretion. Type C CpG ODNs combine features of both types A and B. They contain a complete phosphorothioate backbone and a CpG containing palindromic motif. Type C CpG ODNs induce strong IFN-α production from pDC and B cell stimulation.

Control CpG ODNs that do not stimulate TLR9 have been designed for each stimulatory CpG ODN. They feature the same sequence as their stimulatory counterparts but contain GpC dinucleotides in place of CpG dinucleotides.

Stimulatory CpG ODNs are available labeled with FITC at their 3 terminus. FITC-labeled CpG ODNs are useful to study their cellular uptake and localization by confocal laser-scanning microscopy or flow cytometry.

Unlike mammalian DNA, bacterial DNA is rich in unmethylated CpG motifs and thus activates TLR9. E. coli DNA can be of two types, double-stranded DNA and single-stranded DNA complexed with a cationic lipid. E. coli DNA ef is an ultrapure, endotoxin-free (ef) preparation of E. coli K12 double-stranded DNA devoid of TLR2 and TLR4 activities. E. coli ssDNA is an ultrapure, endotoxin-free preparation of bacterial single-stranded DNA (ssDNA). In E. coli ssDNA, TLR9 binds directly and sequence-specifically to single-stranded unmethylated CpG-DNA. E. coli ssDNA is complexed with the cationic lipid LyoVec™ to allow a better internalization of the immunostimulatory DNA to the acidic compartment where TLR9 is expressed. E. coli DNA ef is an ultrapure, endotoxin-free (ef) preparation of E. coli K12 double-stranded DNA devoid of TLR2 and TLR4 activities.

E. coli DNA ef and E. coli ssDNA are provided lyophilized and shipped at room temperature. Store at −20° C. Lyophilized E. coli DNAs are stable 6 months at −20° C.

Other TLR agonists described in US Application Publication Number 2008/0306050, filed Aug. 17, 2006 and US Application Publication Number 2008/0234251, filed Aug. 17, 2006, are incorporated herein by reference in their entirety.

### Polypeptide Conjugates

Applicants contemplate various peptides and peptide conjugates as agents for use in the methods and compositions of this disclosure. The extracellular domain fragment of the ANXA2 receptor can be used to bind HPV and therefore act as a decoy to inhibit binding of HPV to a cell expressing the ANXA2 receptor. Alternatively or in addition, a peptide containing at least the HPVL2 region can bind the receptor preventing binding of HPV to the ANXA2 receptor. In a further aspect, the agent is SPLI. It is contemplated by the Applicants that these agents can be used alone or in combination with each other known ANXA2 blocking agents.

These can be used in a variety of formulations, which may vary depending on the intended use. For example, one or more can be covalently or non-covalently linked (complexed) to various other molecules, the nature of which may vary depending on the particular purpose. For example, a peptide of the disclosure can be covalently or non-covalently complexed to a macromolecular carrier, including, but not limited to, natural and synthetic polymers, proteins, polysaccharides, polypeptides (amino acids), polyvinyl alcohol, polyvinyl pyrrolidone, and lipids. A peptide can be conjugated to a fatty acid, for introduction into a liposome, see U.S. Pat. No. 5,837,249. A peptide of the disclosure can be complexed covalently or non-covalently with a solid support, a variety of which are known in the art and described herein. An antigenic peptide epitope of the disclosure can be associated with an antigen-presenting matrix such as an MHC complex with or without co-stimulatory molecules.

Examples of protein carriers include, but are not limited to, superantigens, serum albumin, tetanus toxoid, ovalbumin, thyroglobulin, myoglobulin, and immunoglobulin.

Peptide-protein carrier polymers may be formed using conventional cross-linking agents such as carbodimides. Examples of carbodimides are 1-cyclohexyl-3-(2-morpholinyl-(4-ethyl) carbodiimide (CMC), 1-ethyl-3-(3-dimethyaminopropyl) carbodiimide (EDC) and 1-ethyl-3-(4-azonia-44-dimethylpentyl) carbodiimide.

Examples of other suitable cross-linking agents are cyanogen bromide, glutaraldehyde and succinic anhydride. In general, any of a number of homo-bifunctional agents including a homo-bifunctional aldehyde, a homo-bifunctional epoxide, a homo-bifunctional imido-ester, a homo-bifunctional N-hydroxysuccinimide ester, a homo-bifunctional maleimide, a homo-bifunctional alkyl halide, a homo-bifunctional pyridyl disulfide, a homo-bifunctional aryl halide, a homo-bifunctional hydrazide, a homo-bifunctional diazonium derivative and a homo-bifunctional photoreactive compound may be used. Also included are hetero-bifunctional compounds, for example, compounds having an amine-reactive and a sulfhydryl-reactive group, compounds with an amine-reactive and a photoreactive group and compounds with a carbonyl-reactive and a sulfhydryl-reactive group.

Specific examples of such homo-bifunctional cross-linking agents include the bifunctional N-hydroxysuccinimide esters dithiobis(succinimidylpropionate), disuccinimidyl suberate, and disuccinimidyl tartrate; the bifunctional imido-esters dimethyl adipimidate, dimethyl pimelimidate, and dimethyl suberimidate; the bifunctional sulfhydryl-reactive crosslinkers 1,4-di-[3′-(2′-pyridyldithio)propionamido]butane, bismaleimidohexane, and bis-N-maleimido-1,8-octane; the bifunctional aryl halides 1,5-difluoro-2,4-dinitrobenzene and 4,4′-difluoro-3,3′-dinitrophenylsulfone; bifunctional photoreactive agents such as bis-[b-(4-azidosalicylamido)ethyl]disulfide; the bifunctional aldehydes formaldehyde, malondialdehyde, succinaldehyde, glutaraldehyde, and adipaldehyde; a bifunctional epoxide such as 1,4-butaneodiol diglycidyl ether; the bifunctional hydrazides adipic acid dihydrazide, carbohydrazide, and succinic acid dihydrazide; the bifunctional diazoniums o-tolidine, diazotized and bis-diazotized benzidine; the bifunctional alkylhalides N1N′-ethylene-bis(iodoacetamide), N1N′-hexamethylene-bis(iodoacetamide), N1N′-undecamethylene-bis(iodoacetamide), as well as benzylhalides and halomustards, such as a1a′-diiodo-p-xylene sulfonic acid and tri(2-chloroethyl)amine, respectively.

Examples of common hetero-bifunctional cross-linking agents that may be used to effect the conjugation of proteins to peptides include, but are not limited to, SMCC (succinimidyl-4-(N-maleimidomethyl)cyclohexane-1-carboxylate), MBS (m-maleimidobenzoyl-N-hydroxysuccinimide ester), SIAB (N-succinimidyl(4-iodoacteyl)aminobenzoate), SMPB (succinimidyl-4-(p-maleimidophenyl)butyrate), GMBS (N-(γ-maleimidobutyryloxy)succinimide ester), MPBH (4-(4-N-maleimidopohenyl) butyric acid hydrazide), M2C2H (4-(N-maleimidomethyl)cyclohexane-1-carboxyl-hydrazide), SMPT (succinimidyloxycarbonyl-α-methyl-α-(2-pyridyldithio)toluene), and SPDP (N-succinimidyl 3-(2-pyridyldithio)propionate).

Cross-linking may be accomplished by coupling a carbonyl group to an amine group or to a hydrazide group by reductive amination.

Peptides of the disclosure also may be formulated as non-covalent attachment of monomers through ionic, adsorptive, or biospecific interactions. Complexes of peptides with highly positively or negatively charged molecules may be done through salt bridge formation under low ionic strength environments, such as in deionized water. Large complexes can be created using charged polymers such as poly-(L-glutamic acid) or poly-(L-lysine) which contain numerous negative and positive charges, respectively. Adsorption of peptides may be done to surfaces such as microparticle latex beads or to other hydrophobic polymers, forming non-covalently associated peptide-superantigen complexes effectively mimicking cross-linked or chemically polymerized protein. Finally, peptides may be non-covalently linked through the use of biospecific interactions between other molecules. For instance, utilization of the strong affinity of biotin for proteins such as avidin or streptavidin or their derivatives could be used to form peptide complexes. These biotin-binding proteins contain four binding sites that can interact with biotin in solution or be covalently attached to another molecule. (See Wilchek (1988) Anal. Biochem. 171:1-32). Peptides can be modified to possess biotin groups using common biotinylation reagents such as the N-hydroxysuccinimidyl ester of D-biotin (NHS-biotin) which reacts with available amine groups on the protein. Biotinylated peptides then can be incubated with avidin or streptavidin to create large complexes. The molecular mass of such polymers can be regulated through careful control of the molar ratio of biotinylated peptide to avidin or streptavidin.

Also provided by this application are the peptides and polypeptides described herein conjugated to a label, e.g., a fluorescent or bioluminescent label, for use in the diagnostic methods. For example, detectably labeled peptides and polypeptides can be bound to a column and used for the detection and purification of antibodies. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in Haugland, Richard P. (1996) Molecular Probes Handbook.

The polypeptides of this disclosure also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to, Freund's Complete Adjuvant, Freund's Incomplete Adjuvant and mineral salts.

### Therapeutic Antibody Compositions

This disclosure also provides an antibody capable of inhibiting HPV binding to ANXA2 receptor by forming a complex with the ANXA2 receptor. In some embodiments, the antibody is a modified polypeptide of the antibody as described herein. In some embodiments, the antibody is a blocking fragment of the antibody. The term “antibody” includes polyclonal antibodies and monoclonal antibodies, antibody fragments, as well as derivatives thereof. The antibodies include, but are not limited to mouse, rat, goat, and rabbit or human antibodies. Antibodies can be produced in cell culture, in phage, or in various animals, including but not limited to cows, rabbits, goats, mice, rats, hamsters, guinea pigs, sheep, dogs, cats, monkeys, chimpanzees, apes, etc. The antibodies are also useful to identify and purify therapeutic polypeptides.

This disclosure also provides an antibody-peptide complex comprising antibodies described above and a polypeptide that specifically binds to the antibody. In one aspect the polypeptide is the polypeptide against which the antibody was raised. In one aspect the antibody-peptide complex is an isolated complex. In a further aspect, the antibody of the complex is, but not limited to, a polyclonal antibody, a monoclonal antibody, a humanized antibody or an antibody derivative described herein. Either or both of the antibody or peptide of the antibody-peptide complex can be detectably labeled. In one aspect, the antibody-peptide complex of the disclosure can be used as a control or reference sample in diagnostic or screening assays.

Polyclonal antibodies of the disclosure can be generated using conventional techniques known in the art and are well-described in the literature. Several methodologies exist for production of polyclonal antibodies. For example, polyclonal antibodies are typically produced by immunization of a suitable mammal such as, but not limited to, chickens, goats, guinea pigs, hamsters, horses, mice, rats, and rabbits. An antigen is injected into the mammal, which induces the B-lymphocytes to produce IgG immunoglobulins specific for the antigen. This IgG is purified from the mammals serum. Variations of this methodology include modification of adjuvants, routes and site of administration, injection volumes per site and the number of sites per animal for optimal production and humane treatment of the animal. For example, adjuvants typically are used to improve or enhance an immune response to antigens. Most adjuvants provide for an injection site antigen depot, which allows for a slow release of antigen into draining lymph nodes. Other adjuvants include surfactants which promote concentration of protein antigen molecules over a large surface area and immunostimulatory molecules. Non-limiting examples of adjuvants for polyclonal antibody generation include Freund's adjuvants, Ribi adjuvant system, and Titermax. Polyclonal antibodies can be generated using methods described in U.S. Pat. Nos. 7,279,559; 7,119,179; 7,060,800; 6,709,659; 6,656,746; 6,322,788; 5,686,073; and 5,670,153.

The monoclonal antibodies of the disclosure can be generated using conventional hybridoma techniques known in the art and well-described in the literature. For example, a hybridoma is produced by fusing a suitable immortal cell line (e.g., a myeloma cell line such as, but not limited to, Sp2/0, Sp2/0-AG14, NSO, NS1, NS2, AE-1, L.5, >243, P3X63Ag8.653, Sp2 SA3, Sp2 MAI, Sp2 SS1, Sp2 SA5, U397, MLA 144, ACT IV, MOLT4, DA-1, JURKAT, WEHI, K-562, COS, RAJI, NIH 3T3, HL-60, MLA 144, NAMAIWA, NEURO 2A, CHO, PerC.6, YB2/O) or the like, or heteromyelomas, fusion products thereof, or any cell or fusion cell derived therefrom, or any other suitable cell line as known in the art (see, e.g., www.atcc.org, www.lifetech.com, last accessed on Nov. 26, 2007, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. Antibody producing cells can also be obtained from the peripheral blood or, preferably the spleen or lymph nodes, of humans or other suitable animals that have been immunized with the antigen of interest. Any other suitable host cell can also be used for expressing-heterologous or endogenous nucleic acid encoding an antibody, specified fragment or variant thereof, of the present disclosure. The fused cells (hybridomas) or recombinant cells can be isolated using selective culture conditions or other suitable known methods, and cloned by limiting dilution or cell sorting, or other known methods.

In one embodiment, the antibodies described herein can be generated using a Multiple Antigenic Peptide (MAP) system. The MAP system utilizes a peptidyl core of three or seven radially branched lysine residues, on to which the antigen peptides of interest can be built using standard solid-phase chemistry. The lysine core yields the MAP bearing about 4 to 8 copies of the peptide epitope depending on the inner core that generally accounts for less than 10% of total molecular weight. The MAP system does not require a carrier protein for conjugation. The high molar ratio and dense packing of multiple copies of the antigenic epitope in a MAP has been shown to produce strong immunogenic response. This method is described in U.S. Pat. No. 5,229,490 and is herein incorporated by reference in its entirety.

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from various commercial vendors such as Cambridge Antibody Technologies (Cambridgeshire, UK), MorphoSys (Martinsreid/Planegg, Del.), Biovation (Aberdeen, Scotland, UK) BioInvent (Lund, Sweden), using methods known in the art. See U.S. Pat. Nos. 4,704,692; 5,723,323; 5,763,192; 5,814,476; 5,817,483; 5,824,514; 5,976,862. Alternative methods rely upon immunization of transgenic animals (e.g., SCID mice, Nguyen et al. (1977) Microbiol. Immunol. 41:901-907 (1997); Sandhu et al. (1996) Crit. Rev. Biotechnol. 16:95-118; Eren et al. (1998) Immunol. 93:154-161 that are capable of producing a repertoire of human antibodies, as known in the art and/or as described herein. Such techniques, include, but are not limited to, ribosome display (Hanes et al. (1997) Proc. Natl. Acad. Sci. USA 94:4937-4942; Hanes et al. (1998) Proc. Natl. Acad. Sci. USA 95:14130-14135); single cell antibody producing technologies (e.g., selected lymphocyte antibody method (“SLAM”) (U.S. Pat. No. 5,627,052, Wen et al. (1987) J. Immunol. 17:887-892; Babcook et al. (1996) Proc. Natl. Acad. Sci. USA 93:7843-7848); gel microdroplet and flow cytometry (Powell et al. (1990) Biotechnol. 8:333-337; One Cell Systems, (Cambridge, Mass.); Gray et al. (1995) J. Imm. Meth. 182:155-163; and Kenny et al. (1995) Bio. Technol. 13:787-790); B-cell selection (Steenbakkers et al. (1994) Molec. Biol. Reports 19:125-134).

Antibody derivatives of the present disclosure can also be prepared by delivering a polynucleotide encoding an antibody of this disclosure to a suitable host such as to provide transgenic animals or mammals, such as goats, cows, horses, sheep, and the like, that produce such antibodies in their milk. These methods are known in the art and are described for example in U.S. Pat. Nos. 5,827,690; 5,849,992; 4,873,316; 5,849,992; 5,994,616; 5,565,362; and 5,304,489.

The term “antibody derivative” includes post-translational modification to linear polypeptide sequence of the antibody or fragment. For example, U.S. Pat. No. 6,602,684 B1 describes a method for the generation of modified glycol-forms of antibodies, including whole antibody molecules, antibody fragments, or fusion proteins that include a region equivalent to the Fc region of an immunoglobulin, having enhanced Fc-mediated cellular toxicity, and glycoproteins so generated.

Antibody derivatives also can be prepared by delivering a polynucleotide of this disclosure to provide transgenic plants and cultured plant cells (e.g., but not limited to tobacco, maize, and duckweed) that produce such antibodies, specified portions or variants in the plant parts or in cells cultured therefrom. For example, Cramer et al. (1999) Curr. Top. Microbol. Immunol. 240:95-118 and references cited therein, describe the production of transgenic tobacco leaves expressing large amounts of recombinant proteins, e.g., using an inducible promoter. Transgenic maize have been used to express mammalian proteins at commercial production levels, with biological activities equivalent to those produced in other recombinant systems or purified from natural sources. See, e.g., Hood et al. (1999) Adv. Exp. Med. Biol. 464:127-147 and references cited therein. Antibody derivatives have also been produced in large amounts from transgenic plant seeds including antibody fragments, such as single chain antibodies (scFv's), including tobacco seeds and potato tubers. See, e.g., Conrad et al. (1998) Plant Mol. Biol. 38:101-109 and reference cited therein. Thus, antibodies of the present disclosure can also be produced using transgenic plants, according to know methods.

Antibody derivatives also can be produced, for example, by adding exogenous sequences to modify immunogenicity or reduce, enhance or modify binding, affinity, on-rate, off-rate, avidity, specificity, half-life, or any other suitable characteristic. Generally part or all of the non-human or human CDR sequences are maintained while the non-human sequences of the variable and constant regions are replaced with human or other amino acids.

In general, the CDR residues are directly and most substantially involved in influencing antigen binding. Humanization or engineering of antibodies of the present disclosure can be performed using any known method such as, but not limited to, those described in U.S. Pat. Nos. 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; and 4,816,567.

Techniques for making partially to fully human antibodies are known in the art and any such techniques can be used. According to one embodiment, fully human antibody sequences are made in a transgenic mouse which has been engineered to express human heavy and light chain antibody genes. Multiple strains of such transgenic mice have been made which can produce different classes of antibodies. B cells from transgenic mice which are producing a desirable antibody can be fused to make hybridoma cell lines for continuous production of the desired antibody. (See for example, Russel et al. (2000) Infection and Immunity April 2000:1820-1826; Gallo et al. (2000) European J. of Immun. 30:534-540; Green (1999) J. of Immun. Methods 231:11-23; Yang et al. (1999A) J. of Leukocyte Biology 66:401-410; Yang (1999B) Cancer Research 59(6):1236-1243; Jakobovits (1998) Advanced Drug Delivery Reviews 31:33-42; Green and Jakobovits (1998) J. Exp. Med. 188(3):483-495; Jakobovits (1998) Exp. Opin. Invest. Drugs 7(4):607-614; Tsuda et al. (1997) Genomics 42:413-421; Sherman-Gold (1997) Genetic Engineering News 17(14); Mendez et al. (1997) Nature Genetics 15:146-156; Jakobovits (1996) Weir's Handbook of Experimental Immunology, The Integrated Immune System Vol. IV, 194.1-194.7; Jakobovits (1995) Current Opinion in Biotechnology 6:561-566; Mendez et al. (1995) Genomics 26:294-307; Jakobovits (1994) Current Biology 4(8):761-763; Arbones et al. (1994) Immunity 1(4):247-260; Jakobovits (1993) Nature 362(6417):255-258; Jakobovits et al. (1993) Proc. Natl. Acad. Sci. USA 90(6):2551-2555; and U.S. Pat. No. 6,075,181.)

The antibodies of this disclosure also can be modified to create chimeric antibodies. Chimeric antibodies are those in which the various domains of the antibodies' heavy and light chains are coded for by DNA from more than one species. See, e.g., U.S. Pat. No. 4,816,567.

Alternatively, the antibodies of this disclosure can also be modified to create veneered antibodies. Veneered antibodies are those in which the exterior amino acid residues of the antibody of one species are judiciously replaced or “veneered” with those of a second species so that the antibodies of the first species will not be immunogenic in the second species thereby reducing the immunogenicity of the antibody. Since the antigenicity of a protein is primarily dependent on the nature of its surface, the immunogenicity of an antibody could be reduced by replacing the exposed residues which differ from those usually found in another mammalian species antibodies. This judicious replacement of exterior residues should have little, or no, effect on the interior domains, or on the interdomain contacts. Thus, ligand binding properties should be unaffected as a consequence of alterations which are limited to the variable region framework residues. The process is referred to as “veneering” since only the outer surface or skin of the antibody is altered, the supporting residues remain undisturbed.

The procedure for “veneering” makes use of the available sequence data for human antibody variable domains compiled by Kabat et al. (1987) Sequences of Proteins of Immunological Interest, 4th ed., Bethesda, Md., National Institutes of Health, updates to this database, and other accessible U.S. and foreign databases (both nucleic acid and protein). Non-limiting examples of the methods used to generate veneered antibodies include EP 519596; U.S. Pat. No. 6,797,492; and described in Padlan et al. (1991) Mol. Immunol. 28(4-5):489-498.

The term “antibody derivative” also includes “diabodies” which are small antibody fragments with two antigen-binding sites, wherein fragments comprise a heavy chain variable domain (VH) connected to a light chain variable domain (VL) in the same polypeptide chain. (See for example, EP 404,097; WO 93/11161; and Hollinger et al. (1993) Proc. Natl. Acad. Sci. USA 90:6444-6448.) By using a linker that is too short to allow pairing between the two domains on the same chain, the domains are forced to pair with the complementary domains of another chain and create two antigen-binding sites. (See also, U.S. Pat. No. 6,632,926 to Chen et al. which discloses antibody variants that have one or more amino acids inserted into a hypervariable region of the parent antibody and a binding affinity for a target antigen which is at least about two fold stronger than the binding affinity of the parent antibody for the antigen.)

The term “antibody derivative” further includes “linear antibodies”. The procedure for making linear antibodies is known in the art and described in Zapata et al. (1995) Protein Eng. 8(10):1057-1062. Briefly, these antibodies comprise a pair of tandem Fd segments (V_{H}-C_{H}1-VH-C_{H}1) which form a pair of antigen binding regions. Linear antibodies can be bispecific or monospecific.

The antibodies of this disclosure can be recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

Antibodies of the present disclosure include naturally purified products, products of chemical synthetic procedures, and products produced by recombinant techniques from a eukaryotic host, including, for example, yeast, higher plant, insect and mammalian cells, or alternatively from a prokaryotic cells as described above.

If a monoclonal antibody being tested binds with protein or polypeptide, then the antibody being tested and the antibodies provided by the hybridomas of this disclosure are equivalent. It also is possible to determine without undue experimentation, whether an antibody has the same specificity as the monoclonal antibody of this disclosure by determining whether the antibody being tested prevents a monoclonal antibody of this disclosure from binding the protein or polypeptide with which the monoclonal antibody is normally reactive. If the antibody being tested competes with the monoclonal antibody of the disclosure as shown by a decrease in binding by the monoclonal antibody of this disclosure, then it is likely that the two antibodies bind to the same or a closely related epitope. Alternatively, one can pre-incubate the monoclonal antibody of this disclosure with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this disclosure.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Methods 74:307.

The isolation of other hybridomas secreting monoclonal antibodies with the specificity of the monoclonal antibodies of the disclosure can also be accomplished by one of ordinary skill in the art by producing anti-idiotypic antibodies. Herlyn et al. (1986) Science 232:100. An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

In some aspects of this disclosure, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

Antibodies can be labeled with a detectable moiety such as a radioactive atom, a chromophore, a fluorophore, or the like. Such labeled antibodies can be used for diagnostic techniques, either in vivo, or in an isolated test sample. Antibodies can also be conjugated, for example, to a pharmaceutical agent, such as chemotherapeutic drug or a toxin. They can be linked to a cytokine, to a ligand, to another antibody. Suitable agents for coupling to antibodies to achieve an anti-tumor effect include cytokines, such as interleukin 2 (IL-2) and Tumor Necrosis Factor (TNF); photosensitizers, for use in photodynamic therapy, including aluminum (III) phthalocyanine tetrasulfonate, hematoporphyrin, and phthalocyanine; radionuclides, such as iodine-131 (¹³¹I), yttrium-90 (⁹⁰Y), bismuth-212 (²¹²Bi), bismuth-213 (²¹³Bi), technetium-99m (^{99m}Tc), rhenium-186 (¹⁸⁶Re), and rhenium-188 (¹⁸⁸Re); antibiotics, such as doxorubicin, adriamycin, daunorubicin, methotrexate, daunomycin, neocarzinostatin, and carboplatin; bacterial, plant, and other toxins, such as diphtheria toxin, pseudomonas exotoxin A, staphylococcal enterotoxin A, abrin-A toxin, ricin A (deglycosylated ricin A and native ricin A), TGF-alpha toxin, cytotoxin from Chinese cobra (naja naja atra), and gelonin (a plant toxin); ribosome inactivating proteins from plants, bacteria and fungi, such as restrictocin (a ribosome inactivating protein produced by Aspergillus restrictus), saporin (a ribosome inactivating protein from Saponaria officinalis), and RNase; tyrosine kinase inhibitors; ly207702 (a difluorinated purine nucleoside); liposomes containing anti cystic agents (e.g., antisense oligonucleotides, plasmids which encode for toxins, methotrexate, etc.); and other antibodies or antibody fragments, such as F(ab).

The antibodies of the disclosure also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding monoclonal antibodies, or will be able to ascertain such, using routine experimentation.

### siRNA

The agent that inhibits binding of HPV to ANXA2 and/or ANXA/p11 heterotetramer, in some aspects, is an siRNA directed at ANXA2 or p11 or a polynucleotide encoding the siRNA. In one aspect, the siRNA comprises SEQ ID NO: 5. In another aspect, the siRNA comprises SEQ ID NO: 6.

“Short interfering RNAs” (siRNA) refer to double-stranded RNA molecules (dsRNA), generally, from about 10 to about 30 nucleotides in length that are capable of mediating RNA interference (RNAi). “RNA interference” (RNAi) refers to sequence-specific or gene specific suppression of gene expression (protein synthesis) that is mediated by short interfering RNA (siRNA). As used herein, the term siRNA includes short hairpin RNAs (shRNAs). A siRNA directed to a gene or the mRNA of a gene may be a siRNA that recognizes the mRNA of the gene and directs a RNA-induced silencing complex (RISC) to the mRNA, leading to degradation of the mRNA. A siRNA directed to a gene or the mRNA of a gene may also be a siRNA that recognizes the mRNA and inhibits translation of the mRNA. A siRNA may be chemically modified to increase its stability and safety. See, e.g. Dykxhoorn and Lieberman (2006) Annu. Rev. Biomed. Eng. 8:377-402 and U.S. Patent Application Publication No.: 2008/0249055.

“Double stranded RNAs” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

“MicroRNAs” (miRNA) refer to single-stranded RNA molecules of 21-23 nucleotides in length, which regulate gene expression. miRNAs are encoded by genes from whose DNA they are transcribed but miRNAs are not translated into protein (non-coding RNA); instead each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

siRNA to inhibit expression can be designed and delivered following procedures known in the art. See, e.g., Dykxhoorn and Lieberman (2006) Annu. Rev. Biomed. Eng. 8:377-402; Dykxhoorn et al. (2006) Gene Therapy 13:541-52; Aagaard and Rossi (2007) Adv. Drug Delivery Rev. 59:75-86; de Fougerolles et al. (2007) Nature Reviews Drug Discovery 6:443-53; Krueger et al. (2007) Oligonucleotides 17:237-250; U.S. Patent Application Publication No.: 2008/0188430.

### MODES FOR CARRYING OUT THE INVENTION

### Pharmaceutical Compositions

In one aspect, the disclosure provides compositions for use in the methods described herein. In some embodiments, the compositions comprise, or alternatively consist essentially of, or yet further consist of small molecules that inhibit HPV binding to ANXA2 receptor. In another aspect, the compositions comprise, or alternatively consist essentially of, or yet further consist of a peptide, protein or antibody as described above, that inhibits HPV binding to ANXA2 receptor. In an alternate aspect, one or more of the above are combined in a single composition or yet further combined with other known agents such as a TLR agonist or agent that inhibits ANXA2 receptor binding. In one aspect, the agent is SPLI or an agent that enhances expression of SPLI when administered to a subject. In another aspect, the agent is a protein or polypeptide or peptide conjugate comprising, or alternatively consisting essentially of, or yet further consisting of HPV L2 peptide that inhibits binding of HPV to the ANXA2 receptor, an non-limiting example of which is provided in FIG. 3. Additional embodiments include a protein or polypeptide or peptide conjugate comprising, or alternatively consisting essentially of an amino acid sequence identified under Gen Bank Accessions, non-limiting examples of which include numbers AB014925, or AB014918, BAE16268, CAA75467, CAA75460 and CAA75453 (last accessed on Sep. 29, 2009) or those having at least 75%, or alternatively at least 80%, or alternatively at least 85%, or alternatively at least 90%, or alternatively at least 95%, or alternatively at least 97% sequence identity to these amino acid sequences.

In one aspect, the agent comprises, or alternatively consists essentially of, or yet further consists of an amino acid sequence of SPLI, an non-limiting example of which is shown in FIG. 3. Additional embodiments include a protein or polypeptide or peptide conjugate comprising, or alternatively consisting essentially of an amino acid sequence identified under Gen Bank Accessions, non-limiting examples of which include numbers NP_{—}003055, PO3973, CAB64235, AAA60559, EAW75869 and AAH20708, (last accessed on Sep. 29, 2009) or those having at least 75%, or alternatively at least 80%, or alternatively at least 85%, or alternatively at least 90%, or alternatively at least 95%, or alternatively at least 97% sequence identity to these amino acid sequences.

In a further aspect, the agent is an antibody or fragment thereof that specifically recognized and binds either the ANXA2 receptor or HPV, e.g., HPV L2 domain, thereby inhibiting the binding of the virus to the ANXA2 receptor. The antibodies can be monoclonal or polyclonal or fragments thereof including derivatives and modifications thereof as described herein.

The compositions can further comprise a TLR agonist as described herein.

This disclosure further comprises one or more of the agents as described herein. A non-limiting example is a composition comprising, or alternatively consisting essentially of, or yet further consisting of, SLPI and a TLR agonist, each in an effective amount to inhibit HPV infection of LC or tissue containing LC.

In one aspect, the compositions further comprise, or alternatively consist essentially of, or yet further consist of agents selected from the group of inflammatory agent, analgesic, or anti-human immunodeficiency virus (HIV) agent.

An inflammatory agent can be any agent that induces inflammation. Inflammation can be caused by physical ablation of tissue or by injury to a tissue. Inflammation involves infiltration of white blood cells into tissue and phagocytosis by white blood cells and can be accompanied by accumulation of pus and an increase in the local temperature.

A local inflammatory response can be accompanied by systemic changes: fever, malaise, an increase in circulating leukocytes (leukocytosis), and increases in specific circulating proteins called acute-phase reactants. The process of inflammation, both vascular and cellular, can be due to an array of molecules produced locally. These mediators include histamine, leukotrienes, prostaglandins, complement components, kinins, antibodies, and interleukins.

Examples of anti-HIV agents include, but are not limited to, nucleoside and nucleotide reverse transcriptase (RT) inhibitors; non-nucleoside reverse transcriptase inhibitors; protease inhibitors (PIs); viral absorption inhibitors; and viral coreceptor agonists. Examples of nucleoside and nucleotide reverse transcriptase (RT) inhibitors include, but are not limited to, nucleoside analog such as zidovudine; and nucleotide analog. Examples of non-nucleoside reverse transcriptase inhibitors include, but are not limited to, non-nucleoside analog such as, but not limited to, nevirapine, delavirdine, and efavirenz. Examples of PIs include, but are not limited to, HIV protease and ABT-378 or lopinavir. Examples of viral absorption inhibitors include, but are not limited to, Cosalane. Examples of viral coreceptor agonists include, but are not limited to, bicyclams.

Examples of analgesics include, but are not limited to, paracetamol (para-acetylaminophenol, also known in the US as acetaminophen); a non-steroidal anti-inflammatory drugs (NSAIDs) such as, but not limited to, the salicylates; COX-2 inhibitors, such as, but not limited to, rofecoxib and celecoxib; opiates and morphinomimetics such as, but not limited to, morphine, the archetypal opioid, and various other substances (e.g. codeine, oxycodone, hydrocodone, diamorphine, pethidine); and synthetic drugs with narcotic properties such as tramadol, and various others.

In some aspect, the composition further comprises, or alternatively consists essentially of, or yet further consists of a pharmaceutically acceptable carrier. In another aspect, the compositions contain carriers that modulate (controlled release) the release of the active agent when administered to a subject in need thereof. In a further aspect, the compositions are suitable for topical application to the mucosal surface of a subject in need of such treatment.

The pharmaceutical compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil; cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this disclosure are formulated for pharmaceutical administration to a mammal, preferably a human being. Such pharmaceutical compositions of the disclosure may be administered in a variety of ways, preferably topically.

Pharmaceutically acceptable excipients and carriers and dosage forms are generally known to those skilled in the art and are included in the disclosure. It should be understood that a specific dosage and treatment regimen for any particular patient will depend upon a variety of factors, including the activity of the specific antidote employed, the age, body weight, general health, sex and diet, renal and hepatic function of the patient, and the time of administration, rate of excretion, drug combination, judgment of the treating physician or veterinarian and severity of the particular disease being treated.

For prophylactic administration, the compound can be administered to a patient at risk of developing one of the previously described conditions. For example, prophylactic administration can be applied to avoid the onset of symptoms in a patient diagnosed with the underlying disorder such as HPV infection.

The amount of compound administered will depend upon a variety of factors, including, for example, the particular indication being treated, the mode of administration, whether the desired benefit is prophylactic or therapeutic, the severity of the indication being treated and the age and weight of the patient, and the bioavailability of the particular active compound. Determination of an effective dosage is well within the capabilities of those skilled in the art.

Effective dosages can be estimated initially from in vitro assays. For example, an initial dosage for use in animals can be formulated to achieve a local (topical) or circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamagon Press, and the references cited therein.

Initial dosages can also be estimated from in vivo data, such as animal models. Animal models useful for testing the efficacy of compounds to treat or prevent the various diseases described above are known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. For example, the compounds can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the agents and/or compositions will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

To provide for a sustained release of compounds of the disclosure, one or more pH-dependent binders can be chosen to control the dissolution profile of the sustained release formulation so that the formulation releases compound slowly and continuously as the formulation is passed through the stomach and gastrointestinal tract. Accordingly, the pH-dependent binders suitable for use in this disclosure are those which inhibit rapid release of drug from a tablet during its residence in the stomach (where the pH is-below about 4.5), and which promotes the release of a therapeutic amount of the compound of the disclosure from the dosage form in the lower gastrointestinal tract (where the pH is generally greater than about 4.5). Many materials known in the pharmaceutical art as “enteric” binders and coating agents have a desired pH dissolution properties. The examples include phthalic acid derivatives such as the phthalic acid derivatives of vinyl polymers and copolymers, hydroxyalkylcelluloses, alkylcelluloses, cellulose acetates, hydroxyalkylcellulose acetates, cellulose ethers, alkylcellulose acetates, and the partial esters thereof, and polymers and copolymers of lower alkyl acrylic acids and lower alkyl acrylates, and the partial esters thereof. One or more pH-dependent binders present in the sustained release formulation of the disclosure are in an amount ranging from about 1 to about 20 wt %, more preferably from about 5 to about 12 wt % and most preferably about 10 wt %.

One or more pH-independent binders may be in used in oral sustained release formulation of the disclosure. The pH-independent binders can be present in the formulation of this disclosure in an amount ranging from about 1 to about 10 wt %, and preferably in amount ranging from about 1 to about 3 wt % and most preferably about 2 wt %.

The sustained release formulation of the disclosure may also contain pharmaceutical excipients intimately admixed with the compound and the pH-dependent binder. Pharmaceutically acceptable excipients may include, for example, pH-independent binders or film-forming agents such as hydroxypropyl methylcellulose, hydroxypropyl cellulose, methylcellulose, polyvinylpyrrolidone, neutral poly(meth)acrylate esters, starch, gelatin, sugars, carboxymethylcellulose, and the like. Other useful pharmaceutical excipients include diluents such as lactose, mannitol, dry starch, microcrystalline cellulose and the like; surface active agents such as polyoxyethylene sorbitan esters, sorbitan esters and the like; and coloring agents and flavoring agents. Lubricants (such as talc and magnesium stearate) and other tableting aids can also be optionally present.

The sustained release formulations of this disclosure have a TLR agonist of this disclosure in the range of about 50% by weight to about 95% or more by weight, and preferably between about 70% to about 90% by weight; a pH-dependent binder content of between 5% and 40%, preferably between 5% and 25%, and more preferably between 5% and 15%; with the remainder of the dosage form comprising pH-independent binders, fillers, and other optional excipients.

For buccal administration, the compositions may take the form of tablets or lozenges formulated in the conventional manner.

For rectal and vaginal routes of administration, the active compound(s) can be formulated as solutions (for retention enemas), suppositories, or ointments containing conventional suppository bases such as cocoa butter or other glycerides.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device can be accompanied by instructions for administration.

The TLR agonist described herein, or compositions thereof, will generally be used in an amount effective to achieve the intended result, for example, in an amount effective to treat or prevent the particular condition being treated. The TLR agonist(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a TLR agonist to a patient suffering from HPV infection provides therapeutic benefit not only when the HPV infection is eradicated or ameliorated, but also when the patient reports a decrease in the severity or duration of the symptoms associated with the HPV infection. Therapeutic benefit also includes halting or slowing the progression of the disease, regardless of whether improvement is realized.

The amount of TLR agonist administered will depend upon a variety of factors, including, for example, the particular condition being treated, the mode of administration, the severity of the condition being treated, the age and weight of the patient, the bioavailability of the particular active compound. Determination of an effective dosage is well within the capabilities of those skilled in the art. As known by those of skill in the art, the preferred dosage of compounds of the disclosure will also depend on the age, weight, general health, and severity of the condition of the individual being treated. Dosage may also need to be tailored to the sex of the individual and/or the lung capacity of the individual, where administered by inhalation. Dosage, and frequency of administration of the compounds or prodrugs thereof, will also depend on whether the compounds are formulated for treatment of acute episodes of a condition or for the prophylactic treatment of a disorder. A skilled practitioner will be able to determine the optimal dose for a particular individual.

Effective dosages can be estimated initially from in vitro assays. For example, an initial dosage for use in animals can be formulated to achieve a circulating blood or serum concentration of active compound that is at or above an IC50 of the particular TLR agonist as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” GOODMAN AND GILMAN′S THE PHARMACEUTICAL BASIS OF THERAPEUTICS, Chapter 1, pp. 1-46, latest edition, Pergamagon Press, and the references cited therein.

Initial dosages can also be estimated from in vivo data, such as animal models. Animal models useful for testing the efficacy of TLR agonist to treat or prevent the various diseases described above are well-known in the art. Ordinarily skilled artisans can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the TLR agonist, its bioavailability, the mode of administration, and various factors discussed above. Dosage amount and interval can be adjusted individually to provide plasma levels of the compound(s) which are sufficient to maintain therapeutic or prophylactic effect. For example, the TLR agonist can be administered once per week, several times, per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

Preferably, the TLR agonist(s) will provide therapeutic or prophylactic benefit without causing substantial toxicity. Toxicity of the TLR agonist(s) can be determined using standard pharmaceutical procedures. The dose ratio between toxic and therapeutic (or prophylactic) effect is the therapeutic index. The TLR agonist(s) that exhibit high therapeutic indices are preferred.

The foregoing disclosure pertaining to the dosage requirements for the TLR agonist of the disclosure is pertinent to dosages required for prodrugs, with the realization, apparent to the skilled artisan, that the amount of prodrug(s) administered will also depend upon a variety of factors, including, for example, the bioavailability of the particular prodrug(s) and the conversation rate and efficiency into active drug compound under the selected route of administration. Determination of an effective dosage of prodrug(s) for a particular use and mode of administration is well within the capabilities of those skilled in the art. In some embodiments, the topical or oral formulations of TLR agonists are within the range of about 1-10%·wt/vol. In some embodiments, the non-topical formulations of TLR agonists are within the range of about 500-1500 microgram per injection.

### Therapeutic, Diagnostic and Screening Utilities

This disclosure provides a method for inhibiting HPV infection of LC or tissue containing LC comprising, or alternatively consisting essentially of, or yet further consisting of, administering to the LC or tissue an effective amount of an agent that inhibits HPV binding to annexin A2 (ANXA2) present on the surface of the cell, thereby inhibiting HPV infection. In one aspect, the agent is the active agent of the compositions as described above. In one aspect, the agent is SLPI. In another aspect, the agent is an agent that upregulates the expression of SLPI in the tissue. In another aspect, the agent is administered in combination with a TLR agonist. The second agent can be co-administered or administered prior to or subsequent to administration of the agent that inhibits HPV infection.

In another aspect, this disclosure provides a method for treating or preventing HPV infection in a subject having or at risk of HPV infection, comprising, or alternatively consisting essentially of, or yet further consisting of, administering to the subject an effective amount of an agent that inhibits HPV binding to ANXA2/p11 heterotetramer, thereby preventing or treating HPV infection in the subject. In one aspect, the agent is SLPI. In another aspect, the agent is an agent that upregulates the expression of SLPI in the subject. In another aspect, the agent is administered in combination with a TLR agonist. The second agent can be co-administered or administered prior to or subsequent to administration of the agent that inhibits HPV infection.

In another aspect, this disclosure provides a method for preventing or inhibiting HPV-related pathologies in a subject having or at risk of an HPV infection, comprising, or alternatively consisting essentially of, or yet further consisting of, administering to the subject an effective amount of an agent that inhibits HPV binding to ANXA2/p11 heterotetramer, thereby preventing or treating HPV-related pathologies in the subject. In one aspect, the agent is SLPI. In another aspect, the agent is an agent that upregulates the expression of SLPI in the subject. In another aspect, the agent is administered in combination with a TLR agonist. The second agent can be co-administered or administered prior to or subsequent to administration of the agent that prevents or inhibits HPV infection.

For each of the above noted methods, the agents for use in the methods include the active agents as provided in the compositions as described above.

In one aspect, the agent for use in the above noted methods are administered alone or in as a composition. Compositions described above, can be administered to the subject in need of. In one aspect, the composition is directly delivered onto skin or mucosa.

The methods are useful to treat a subject in need, thereof, e.g., an animal or human. When administered to an animal, it can be used as an animal model to study disease or test for additional new agents or therapies that can be used alone or in combination with the compositions and therapies as described herein. Subjects in need of such therapy include those at risk for HPV infection or have an HPV infection or those who are immune compromised due to the existence of another disease.

In one aspect, the composition and methods of this disclosure can be used to treat a condition in a subject such as a mammalian subject in need of. In some embodiments, the condition comprises HPV infection, chronic HPV infection or cervical cancer caused by HPV infection.

In another aspect, the composition can be co-administrated, or administered prior to or after administration of a second agent that inhibit HPV infection or enhances SLPI expression or yet further, localized immune response. In some embodiments, the agents are administered in one or more slow release excipients. When more than one is used, the excipients may further have two different rates of release where the composition of the disclosure is released over the course of a few hours, a day or more, followed by several days of release of the second agent. In another aspect, time release encapsulation comprising the compositions of the disclosure can be included in shampoo for convenient administration.

In one aspect, the agents for use in the methods herein, are used in combination with another therapy selected from the group consisting of inflammatory agent, analgesic, or anti-human immunodeficiency virus (HIV) agent.

An inflammatory agent can be any agent that induces inflammation. Inflammation can be caused by physical ablation of tissue or by injury to a tissue. Inflammation involves phagocytosis by white blood cells and can be accompanied by accumulation of pus and an increase in the local temperature.

A local inflammatory response can be accompanied by systemic changes: fever, malaise, an increase in circulating leukocytes (leukocytosis), and increases in specific circulating proteins called acute-phase reactants. The process of inflammation, both vascular and cellular, can be due to an array of molecules produced locally. These mediators include histamine, leukotrienes, prostaglandins, complement components, kinins, antibodies, and interleukins.

Examples of anti-HIV agents include, but are not limited to, nucleoside and nucleotide reverse transcriptase (RT) inhibitors; non-nucleoside reverse transcriptase inhibitors; protease inhibitors (PIs); viral absorption inhibitors; and viral coreceptor agonists. Examples of nucleoside and nucleotide reverse transcriptase (RT) inhibitors include, but are not limited to, nucleoside analog such as zidovudine; and nucleotide analog. Examples of non-nucleoside reverse transcriptase inhibitors include, but are not limited to, non-nucleoside analog such as, but not limited to, nevirapine, delavirdine, and efavirenz. Examples of PIs include, but are not limited to, HIV protease and ABT-378 or lopinavir. Examples of viral absorption inhibitors include, but are not limited to, Cosalane. Examples of viral coreceptor agonists include, but are not limited to, bicyclams.

Examples of analgesics include, but are not limited to, paracetamol (para-acetylaminophenol, also known in the US as acetaminophen); a non-steroidal anti-inflammatory drugs (NSAIDs) such as, but not limited to, the salicylates; COX-2 inhibitors, such as, but not limited to, rofecoxib and celecoxib; opiates and morphinomimetics such as, but not limited to, morphine, the archetypal opioid, and various other substances (e.g. codeine, oxycodone, hydrocodone, diamorphine, pethidine); and synthetic drugs with narcotic properties such as tramadol, and various others.

The compositions can be combined with or coated on contraceptives such as condoms or cervical caps, similar to the use of spermicidal coatings, with the possibility of reducing the transmission of HPV and other viruses like HIV that use ANXA2/p11 heterotetramer as a (co-) receptor for entering cells.

In yet another aspect, having identified the HPV receptor, this disclosure provides a method to design anti-viral (e.g., HPV drugs) or anti-cancer agents that harbor HPV as they can be delivered through the same receptor that HPV utilizes. The method would, in one aspect, determine if a test agent is suitable for inhibiting or preventing receptor binding or an agent such as a virus, or alternatively preventing HPV infection of a LC or tissue containing LC comprising, or alternatively consisting essentially of, or yet further consisting of, (a) administering to a first tissue sample an amount of the test agent; (b) administering to a second tissue sample an effective amount of an agent that inhibits binding of HPV or other agent believed to bind to ANXA2; and (c) comparing the binding of HPV in the first tissue sample to the binding in the second tissue sample and/or third tissue sample, wherein the test agent is suitable for inhibiting or preventing HPV infection if the HPV viral titer of the first tissue sample is similar to the second tissue sample.

Alternatively, the method is practiced by: (a) administering to the first tissue an agent you wish to enter through the ANXA2 receptor; (b) administering to the second tissue the HPV or other agent that binds to ANXA2; and (c) comparing the binding of the first agent to the HPV; wherein the agent can utilize the ANXA2 receptor has similar binding efficiency a the HPV agent or other agent that is known to ANXA2. As noted above, these methods can be used to identify anti-viral or anti-cancer drugs or biologics that harbor HPV as they can be delivered through the same receptor that HPV is using. As is apparent to the skilled artisan, the above methods can be modified to co-administer other drugs or agents to identify or confirm combination therapies.

The agents and compositions of the present disclosure can be used in the manufacture of medicaments and for the treatment of humans and other animals by administration in accordance with conventional procedures, such as an active ingredient in pharmaceutical compositions.

### Kits

In yet another aspect, this disclosure provides a kit for preventing or inhibiting HPV infection in a LC or tissue containing LC or a subject at risk of or having an HPV infection, comprising, or alternatively consisting essentially of, or yet further consisting of, an effective amount of an agent that inhibits HPV binding to ANXA2 in a pharmaceutically acceptable carrier and instructions for use in preventing or inhibiting HPV infection. In one aspect, the agent is SLPI. In another aspect, the agent is an agent that upregulates the expression of SLPI in the subject. In another aspect, the agent is administered in combination with a TLR agonist. The second agent can be co-administered or administered prior to or subsequent to administration of the agent that prevents or inhibits HPV infection.

In some embodiments, the pharmaceutically acceptable carrier in the kits is suitable for topical administration of the agent. Additional agents can be co-formulated or delivered concomitantly or sequentially with the above noted agents, as described herein. The formulations can be for immediate or controlled release of the active ingredients.

In some embodiments, the pharmaceutically acceptable carrier further comprises a penetration or permeation enhancer.

Also provided are kits for administration of the compounds for treatment of disorders as described herein. Kits may further comprise suitable packaging and/or instructions for use of the compound. Kits may also comprise a means for the delivery of the at least one agent or composition and instructions for administration. Alternatively, the kit provides the compound and reagents to prepare a composition for administration. The composition can be in a dry or lyophilized form or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, a tampon or intravaginal device or an intrarectal device.

The kits may include other therapeutic compounds for use in conjunction with the compounds described herein. These compounds can be provided in a separate form or mixed with the compounds of the present disclosure.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the disclosure, kits for treating an individual who suffers from or is susceptible to the conditions described herein are provided, comprising a container comprising a dosage amount of a composition, as disclosed herein, and instructions for use. The container can be any of those known in the art and appropriate for storage and delivery of oral, intravenous, intravaginally, anal, topical, rectal, urethral, or inhaled formulations.

Kits may also be provided that contain sufficient dosages of the effective composition or compound to provide effective treatment for an individual for an extended period, such as a week, 2 weeks, 3, weeks, 4 weeks, 6 weeks, or 8 weeks or more.

The following examples are provided to illustrate select embodiments of the disclosure as disclosed and claimed herein.

### EXPERIMENTAL

Individuals infected with human immunodeficiency virus (HIV-1) have a higher prevalence of human papillomavirus (HPV) infection and a 5-fold increased incidence of HPV-related cancers due to impaired T cell function. Cervical and anal cancers are caused by persistent infection with high-risk oncogenic HPV genotypes. Currently, there is no treatment for persistent HPV infection. Because HPV-related cancers are so prevalent in HIV-infected individuals, there is an urgent need to develop strategies to reduce the risk and prevent the development of HPV-associated malignancies.

Mucosal immunity is essential to combat pathogens that infect the respiratory and genital tracts. Secretory leukocyte protease inhibitor (SLPI), a serine protease inhibitor found in mucosal fluids, plays multiple roles in mucosal immunity due to its anti-protease and anti-microbial activity. SLPI blocks HIV'infection of macrophages by interrupting the interaction of HIV to the annexin A2 (ANXA2) receptor. Langerhans cells (LC) also express ANXA2 and as the initial cellular targets of HIV, play a significant role in viral dissemination. HPV also interacts with LC, the resident antigen presenting cells of the suprabasal mucosa. Applicants have previously shown that the interaction of HPV16 with LC inhibits their maturation, preventing the induction of HPV-specific T cell responses despite the presentation of viral antigens by LC. Applicants also now show that L2 interacts with ANXA2 on LC and this interaction can be blocked by SLPI, similar to HIV infection of macrophages. Without being bound by theory, this observation suggests that SLPI may play a role in altering susceptibility to HPV infection, linking these two viral pathogens to the same receptor.

### HPV Immune Escape

Persistence of a high-risk HPV infection is a major risk factor in the development of cervical cancer. While a majority of women (and likely men) infected with HPV clear the virus, the time taken to do so can range from many months to years (Leggatt et al. (2007) Current Opinion of Immunology 19:232-238). About 15% of HIV negative women that have high-risk HPV infections do not initiate an effective immune response against HPV, allowing the virus to persist for decades. In HIV positive individuals, viral persistence is increased, likely due to a suppressed immune system in addition to viral immune escape.

HPV has developed a variety of escape mechanisms that circumvent immediate elimination, allowing viral replication and persistence in the host. Applicants have shown that HPV manipulates human LC as a mechanism of immune escape. LC located in the epithelial layer of the skin and mucosa are the first and critical antigen presenting cell (APC) to come into contact with HPV. Consequently, LCs are responsible for initiating an effective immune response against HPV infection. Upon recognition of a foreign antigen, LC undergo maturation, which consists of phenotypic and functional changes including up-regulation of co-stimulatory molecules CD80 and CD86, MHC class I and II, chemokine receptors such as CCR7, secretion of cytokines and chemokines, and migration to regional lymph nodes where T cell activation takes place.

Applicants have previously shown that HPV16 L1L2 virus-like particles (VLP), which are morphologically similar to authentic virions, suppress the activation of Langerhans cells (LC) through phosphoinositide-3-kinase (PI3K) activation and Akt inactivation (Fausch et al. (2003) Cancer Res. 63:3478-3482; Fausch et al. (2002) J. Immunol. 169:3242-3249; Fausch et al. (2005) J. Immunol. 174:7172-7178). LC exposed to HPV16 L1 L2 VLP do not up-regulate co-stimulatory molecules and chemokine receptors, do not secrete cytokines and chemokines and do not initiate epitope-specific immune responses against HPV16 VLP-derived antigens (Fausch et al. (2002) J. Immunol. 169:3242-3249). HPV16 L1 L2 VLP-exposed LC present HPV peptides in the absence of co-stimulation, thereby becoming potentially immune-suppressive. This in turn may lead to persistence of the HPV infection and an increased likelihood of cancer development. Prior to Applicants' invention, the interaction between the suppressive phenotype of LC and a cell surface receptor and the consequence for viral immune escape and whether other HPV genotypes also suppress LC activation through ANXA2 was not known. Applicants' elucidation of this information provides a new target for treatment of HPV-associated diseases.

### The Role of SLPI in Innate Anti-Viral Immunity:

Secretory leukocyte protease inhibitor (SLPI) is a natural ligand for ANXA2 and is a hormonally regulated protein produced by many cell types including epithelial cells, mast cells, neutrophils, macrophages and dendritic cells. It is found in mucosal fluids, including cervicovaginal secretions and saliva. Initially identified as a protease inhibitor, it has since been shown to possess multiple distinct properties that protect the host from infection and injury. SLPI possesses anti-microbial, anti-viral, anti-inflammatory, immune-modulatory and wound healing properties in addition to its function as an anti-protease. Accumulating evidence shows that SLPI can influence host cell-pathogen interactions in the mucosa and plays a key role in protecting mucosal surfaces against viral infection. Lower SLPI levels could therefore leave the mucosa more susceptible for infection by pathogens. For example, low levels of SLPI are found in individuals with chronic obstructive pulmonary disease and in females with repeated lower genital tract infections (Draper et al. (2000) American Journal of Obstetrics and Gynecology 183:1243-1248). Low SLPI levels are also associated with increased vertical HIV transmission rates from HIV-infected women to their babies during childbirth and breastfeeding (Farquhar et al. (2002) The Journal of Infectious Diseases 186:1173-1176; Pillay et al. (2001) The Journal of Infectious Diseases 183:653-656). SLPI plays an important role in preventing the transmission of HIV by interrupting the interaction of HIV to the ANXA2 co-receptor on the host cell on macrophages and CD4⁺ T cells (Ma et al. (2004) J. Exp. Med. 200:1337-1346; McNeely et al. (1995) The Journal of Clinical Investigation 96:456-464). Pathogen infection can also alter mucosal SLPI levels. Herpes simplex virus (HSV) evades innate immunity by decreasing SLPI expression (Fakioglu et al. (2008) Journal of Virology 82:9337-9344). However, HIV-1 gp120 has been shown to increase SLPI expression in oral epithelial cells, suggesting that resistance to infection of the oral mucosa by HIV might be due to high levels of SLPI (Jana et al. (2005) Journal of Virology 79:6432-6440).

### Targeting of a Shared HPV and HIV Co-Receptor

ANXA2 is a host cell membrane protein found at the cell surface and is expressed on skin and cervical epithelial cells, macrophages, monocytes and LC. ANXA2 has been associated with CMV infection, identified as a co-factor for HIV infection in macrophages, and is a receptor for respiratory syncytial virus. ANXA2 heterotetramers are associated with the PI3K signal transduction cascade, a pathway that Applicants have found is involved in HPV immune escape (Fausch et al. (2005) J. Immunol. 174:7172-7178). Applicants have shown that the L2 protein of HPV16 interacts with ANXA2.

### Boosting Innate Immunity Against HPV Infection

Differential expression of TLR on innate immune cell subsets has been shown to correlate with induction of different types and quantities of cytokines by specific agonists (Zarember and Godowski (2002) J. Immunol. 168:554-561). Of these, TLR-3, -7 and -8 have been shown to be involved in mediating anti-viral immunity (Koyama et al. (2008) Cytokine 43:336-341). However, for LC, the pattern of TLR expression is controversial and TLR-signaling pathways have not been well characterized. It is not known which TLR pathway in LC would be most amenable to modulation, and a clinically usable TLR agonist that reverses LC-mediated immune suppression has not been identified thus far. TLR agonists may also increase SLPI production, and therefore these compounds would be used to serve a dual purpose in preventing future infection/transmission of HPV/HIV and activating the immune system against these viruses.

HPV is a pathogen that clearly plays a causative role in the development of anogenital cancers, especially in immune compromised people.

Despite AIDS awareness programs and increased routine testing in the last 25 years of the HIV epidemic, new HIV infection continues to occur in the U.S. at a rate of 22.8 per 100,000 people per year and the prevalence rate of HIV/AIDS has not declined in the last several years (Hall et al. (2008) Jama 300:520-529). More than 1 million people in the U.S. and more than 40 million worldwide are currently living with HIV or HIV/AIDS. Up to 25% of HIV-infected people are unaware of their infection, resulting in continued transmission of this virus. HPV infection is rampant in humans, is linked to a multitude of warts, lesions and cancers, and causes significant disease in HIV-infected individuals who are at a higher risk for developing cancer. Greater than 60% of HIV-infected women and 75% of HIV-infected men have concomitant HPV infection that has the potential to develop into cancer. Thus, millions of people worldwide have the potential to benefit from a pan-HPV therapeutic that would eliminate their HPV infections or early precancerous lesions.

### Example 1

HPV capsids are composed of the major L1 capsid protein and the minor L2 capsid protein. HPV16 L1 only VLP activate LC whereas L1L2 VLP (similar to authentic virions) suppress LC activation, suggesting that this effect is mediated by the presence of the L2 capsid protein (our unpublished data). Furthermore, a L2₁₀₈₋₁₂₀ peptide, representing the part of L2 exposed on the capsid surface, inhibits the binding of HPV16 L1L2 VLP to LC and the Applicants have shown that this peptide binds to ANXA2. However, whether ANXA2 mediates entry of HPV16 into LC and epithelial cells and whether the ANXA2-L2 interaction mediates immune suppression of LC is unknown. SLPI is known to bind to ANXA2 and inhibit the entry of HIV on macrophages, therefore it may also block HPV uptake by LC and subsequent immune suppression as well as the interaction of HIV with LC. HPV virions infect basal cells of the epidermis and ANXA2 is expressed by basal cells. Whether SLPI can inhibit binding and entry of HIV and HPV16 on both LC and epithelial cells is unknown and will be examined. Knowledge about the specific interaction between HPV and its cell surface receptor on LC will direct development of strategies to inhibit the interaction with LC that leads to immune escape. If ANXA2 is also found to be an HPV receptor on epithelial cells, these strategies could also be used to prevent recurrent or active HPV infection by inhibiting the binding of HPV to basal epithelial cells. Moreover, strategies that inhibit HIV from interacting with LC might also prevent ongoing transfer of HIV from mucosal surfaces to T cells. Therefore, understanding the interaction between cells of the mucosal immune system, HPV and HIV will provide valuable knowledge for developing strategies to inhibit transmission and clear HPV infections before they cause lesions.

As HPV only infects human beings, human cells are used to study the interaction of HPV with LC and epithelial cells. Primary LCs are isolated from human skin become activated through the migration process and express high levels of MHC and co-stimulatory molecules. Therefore, it is not feasible to isolate un-activated skin-derived LC from human donors with which to conduct functional activation studies. Circulating monocytes are direct precursors of epidermal LC in vivo. Applicants and others have shown that monocyte-derived LC express the same surface markers as epidermal LC (Langerin, E-cadherin, CD11c, CD1a, and intracellular Birbeck granules) (Fausch et al. (2002) J. Immunol. 169:3242-3249) and can be used consistently for functional studies. Thus, for these experiments, immature human LC are generated ex vivo from peripheral blood monocytes isolated from healthy donors using the differentiating cytokines GM-CSF, IL-4 and TGFβ. High numbers of peripheral blood leukocytes will be isolated via leukapheresis of healthy men or women blood donors. Epithelial cells that are used are commercially available primary neonatal foreskin keratinocytes, the commonly used HaCaT epithelial cell line and the HPV16⁺ Caski cervical cancer cell line.

### Inhibition of ANXA2-Mediated Uptake of HPV by SLPI.

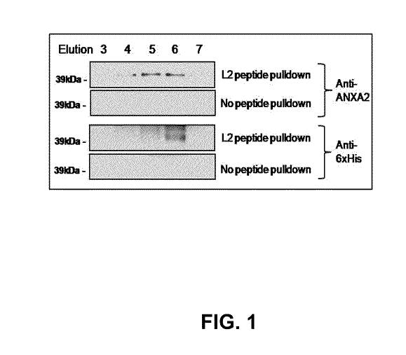

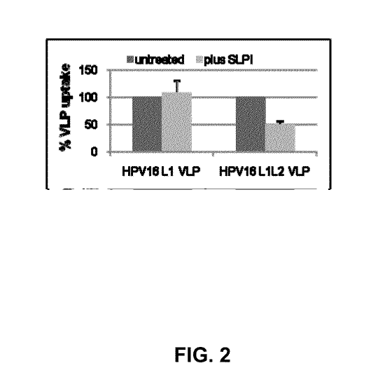

Applicants have shown that HPV16 suppresses the immune response by inhibiting the activation of LC and that the HPV16 L2 protein mediates immune escape. In order to determine the mechanism by which L2 mediates immune suppression, the proteins on the surface of LC that interact with L2 were examined by immunoprecipitation and mass spectrometry. Applicants found that ANXA2 interacts with L2 by immunoprecipitation with a 6×His-tagged L2 peptide and immunoblotting the eluate fractions with an anti-ANXA2 antibody. A clear band corresponding to ˜40 kDa, the size of an ANXA2 monomer, was detected in the same fractions that the His-tagged L2 peptide was detected (FIG. 1). Applicants also found that recombinant human SLPI, a ligand for ANXA2, inhibits uptake of HPV16 L1L2 VLP by LC (FIG. 2).

### Example 2

This example demonstrates that the N-terminus of the HPV minor capsid protein L2 associates with the annexin A2 heterotetramer on the Langerhans cell surface. Inhibiting the interaction between the HPV L2 capsid protein and the annexin A2 heterotetramer or downregulating annexin A2 heterotetramer expression disrupts the internalization of HPV by Langerhans cells, indicating that the annexin A2 heterotetramer is an uptake receptor for HPV. This result is surprising because neither a specific receptor for the HPV L2 protein nor an uptake receptor for HPV has been identified prior to the current invention.

### Materials and Methods

Antibodies. The following antibodies were used in this example: mouse-anti-annexin A2 (BD Biosciences); mouse-anti-annexin A2 light chain (p11) (BD Biosciences); anti-GAPDH (Chemicon) and anti-mouse-IgG-HRP (BD Biosciences).

LC Generation. Human PBL from healthy donors were obtained by leukapheresis (Fausch et al. (2002) J. Immunol. 169: 3242-9). LC and DC were generated from human PBL as previously described (Fahey et al. (2009) J. Immunol. 182: 2919-2928). HPV serology of all donors was negative. All human studies were approved by the USC Institutional Review Board and informed consent was obtained from all donors.

Virus-Like Particles. HPV16L1 VLP and HPV16L1L2 VLP were produced as previously described (Fausch et al. (2002) J. Immunol. 169: 3242-9). Western blot analyses confirmed the presence of L1 and L2 while an ELISA and transmission electron microscopy confirmed the presence of intact particles. An E-toxate kit (Sigma-Aldrich) was used to semi-quantitate endotoxin. The endotoxin level in the preparations was less than 0.06 endotoxin units/ml and this level does not activate LC (Fausch et al. (2002) J. Immunol. 169: 3242-9). Baculovirus DNA used in VLP production procedure does not activate LC (Fausch et al. (2002) J. Immunol. 169: 3242-9).

HPV16 VLP Uptake Assay with Confocal Microscopy. HPV16L1 VLP were labeled with the Alexa Fluor 546 Protein Labeling Kit (Invitrogen) and HPV16L1L2 VLP were labeled with the Alexa Fluor 488 Protein Labeling Kit (Invitrogen) as described in manufacturer's instructions. After collection of the labeled VLP, a Bradford assay was performed to quantify the amount of protein in each VLP prep. LC were incubated with 0.1 μg VLP/10⁶ cells of both AF546-HPV16L1 VLP and AF488-HPV16L1L2 VLP simultaneously in 400 μl PBS in amber 1.5 ml microcentrifuge tubes at 37° C. At each time point (2.5, 5, 15 and 30 min), 400 μl of cold 4% paraformaldehyde was added to the LC to fix the cell and freeze cellular uptake. Cells were subsequently spun down at 800×g for 5 min at 4° C. and then resuspended in DAPI staining buffer (Invitrogen). After 15 min incubation at room temperature, cells were spun down and resuspended in PBS. Cells were visualize by confocal microscopy at the USC Cell and Tissue Imaging Core (Los Angeles, Calif.). As a control, the fluorescent dye was switched for the L1 and L1L2 VLP to ensure the result was not due to the particular dye. Similar results were obtained when the dye was switched.

HPV16L1L2 VLP Binding Assay. LC were incubated with the HPV16 L2 peptide aa 108-120 (LVEETSFIDAGAP SEQ ID NO: 4) (Kawana et al. (2001) J. Virol. 75: 2331-6) at varying concentrations (1-100 μg)/0.5×10⁶ cells for 1 h at 4° C. Subsequently, the LC were incubated with 0.25 μg of HPV16L1L2 VLP/treatment for 1 h at 4° C. and then incubated with an anti-L1 (H16.V5) antibody (a gift from Neil Christensen, Penn State University) at a concentration of 1:25,000 for 30 min at 4° C. The cells were then incubated with biotinylated anti-mouse-IgG2b at a concentration of 1:50 for 30 min at 4° C. Next, the HPV16L1L2 VLP/anti-L1/biotin treated cells were stained with streptavidin-FITC at a concentration of 1:50 for 30 min. The cells were washed between each incubation with 135 μl FACS buffer. In control experiments, cells were left untreated or probed with either peptide/anti-L1/biotin-strepavidin-FITC or VLP/anti-L1/biotin-strepavidin-FITC. Finally, HPV16L1L2 VLP binding to LC was assessed by flow cytometry. Binding is calculated as the MFI of the sample divided by the MFI of the untreated control multiplied by 100.

L2₁₀₈₋₁₂₀ Peptide Pulldown Assay. LC were harvested, washed with PBS, and aliquoted into 1.5 ml microcentrifuge tubes in PBS. Then (6×)His-L2₁₀₈₋₁₂₀ peptide was added to the LC, at a concentration of 50 μg/0.5×10⁶ cells, and incubated for 1 h. In control experiments, LC were left untreated (no peptide added) but exposed to each condition thereafter. Following the incubation, DTSSP (final concentration of 1.5 mM) was added to the LC and incubated for 2 h to cross-link the peptide to the receptor. After the cross-linking reaction was quench with 1 M Tris, LC were washed with PBS and resuspended in and incubated with a bursting solution (10 mM Hepes, 2 mM MgCl, 10 mM KCl₂, 0.05% Tween-20, and Halt Protease Inhibitor Cocktail) for 20 min. Next, the cells were centrifuged for 30 min at 13,000 g. The supernatants were decanted and LC were resuspended in lysis buffer (50 mM NaH₂PO₄, 300 mM NaCl, 50 mM imidazole, 0.05% Tween-20, and Halt Protease Inhibitor Cocktail, pH 8.0). The cells were snap frozen, allowed to thaw, incubated on ice for 30 min, and sonicated for 10 s. Subsequently, the lysates were centrifuged for 30 min at 10,000 g. The lysate supernatants were decanted, mixed with 50% Ni-NTA agarose slurry (Qiagen) and incubated overnight. The following day a column (Thermo Scientific) was assembled to elute the proteins from the Ni-NTA agarose slurry. Once the column was assembled, the lysate-Ni-NTA agarose slurry was washed twice with wash buffer (50 mM NaH₂PO₄, 300 mM NaCl, 50 mM imidazole, and 0.05% Tween-20, pH 8.0). The proteins associated with the Ni-NTA agarose were eluted over 10 fractions. The first fraction was eluted using elution buffer #1 (50 mM NaH₂PO₄, 300 mM NaCl, 100 mM imidazole, and 0.05% Tween-20, pH 8.0). Fractions 2-10 were eluted using elution buffer #2 (50 mM NaH₂PO₄, 300 mM NaCl, 250 mM imidazole, and 0.05% Tween-20, pH 8.0). The eluates were collected and analyzed by non-reducing immunoblots and reduced silver stain gels. Each step of the pulldown assay was performed at 4° C. For the immunoblots, reduced eluates were run on 10% Bis-Tris gels using the NuPAGE Electrophoresis System according to manufacturer's instructions. The protein was then transferred to nitrocellulose, blocked with StartingBlock Blocking Buffer and the membranes were probed with either an anti-ANXA2 antibody (BD Biosciences) or an anti-ANXA2 light chain antibody (BD Biosciences). Blots were subsequently incubated with an HRP-labeled anti-mouse antibody and developed using the Supersignal West Pico chemiluminescent substrate (Thermo Scientific). 10% Tris-HCL gels (Bio-Rad) were used to separate out reduced eluates for silver stain analysis.

Silver Staining Assay. The gel was fixed overnight in 250 ml of fixing solution (50% MetOH, 12% acetic acid, and 0.05% formaldehyde). After fixation, the gel was washed three times with 35% EtOH for 20 min and washed twice with double distilled water. Following the washing, the gel was sensitized using 250 ml of sensitization buffer (100 mM Na₂S₂O₃, 30 mM FeK₃(CN)₆ for 2 min. Next, the gel was washed four times with double distilled water and stained for 20 min with 200 ml of staining solution (0.2% AgNO₃, 0.076% formaldehyde). After the gel was stained it was washed twice with double distilled water and developed to desired darkness with 250 ml of developing solution (6% Na₂CO₃, 0.05% formaldehyde, 0.0004% Na₂S₂O₃). When the gel was developed to the desired darkness the stain was stopped using 50% MetOH/12% acetic acid for 5 min. Specific bands were excised and analyzed by mass spectrometry protein sequencing (Thermo LTQ-ETD mass spectrometer, Thermo Scientific) at the USC proteomics core (Los Angeles, Calif.).

HPV16 VLP Uptake Assay with SLPI. HPV16L1L2 VLP and HPV16L1 VLP were labeled with CFDA-SE using Vybrant CFDA-SE cell tracer kit (Invitrogen) as directed by the manufacturer's instructions. After labeling the HPV16 VLP were dialyzed against 4 liters of cold PBS/0.5 M NaCl to remove all the excess free dye. LC were harvested, washed with PBS, and aliquoted at a concentration of 1×10⁶ cells/200 μl cold PBS into 1.5 ml amber tubes. Subsequently the cells were either left untreated or incubated with increasing concentrations (5-30 μg/ml) of rhu-SLPI (R&D Systems) for 1 h at 4° C. Following the incubation the cells were washed with 500 μl cold PBS and spun down at 800 g for 5 min at 4° C. The supernatant was removed and the LC were resuspended in 400 μl of room temperature FACS buffer. Next, CFDA-SE labeled HPV16L1L2 VLP or HPV16L1 VLP (1 μg/1×10⁶) were incubated with the LC at 37° C. After 15 min, LC were harvested and fixed in 2% paraformaldehyde. Finally, HPV16 VLP uptake by LC was assessed via flow cytometry. Percent uptake is calculated as the mean fluorescent intensity (MFI) of the sample divided by the MFI of the untreated control multiplied by 100.

siRNA Inhibition of ANXA2 in LC and HPV16 VLP Uptake Assay. ANXA2 siRNA was synthesized at the USC Genomics Core (Los Angeles, Calif.) based on the sequences: ANXA2 siRNA #1 (GCAAGUCCCUGUACUAUUATT)/(UAAUAGUACAGGGACUUGCTT) (SEQ ID NO: 5); ANXA2 siRNA #2 (CGGGAUGCUUUGAACAUUGAATT)/(UUCAAUGUUCAAAGCAUCCCGTT) (SEQ ID NO: 6). Control siRNA was from (S76). ANXA2 or Control siRNA was transfected into LC using the Amaxa Human Dendritic Cell Nucleofector kit (Lonza) as directed by the manufacturer's instructions. The cells were incubated for 6 days before they were analyzed by an anti-ANXA2 immunoblot and used in an HPV16 VLP uptake assay. HPV VLP were labeled with CFDA-SE as described above. CFDA-SE labeled HPV16L1L2 VLP or HPV16L1 VLP (1 μg/1×10⁶) were incubated with the nucleofected LC at 37° C. After 15 min, LC were harvested and fixed in 2% paraformaldehyde. Finally, HPV16 VLP uptake by LC was assessed via flow cytometry.

Statistical Analysis. All statistical analyses were performed using GraphPad Prism (GraphPad Software Inc., San Diego, Calif.).

### Differential Uptake Pathways of HPV16L1 and HPV16L1L2 VLP in LC

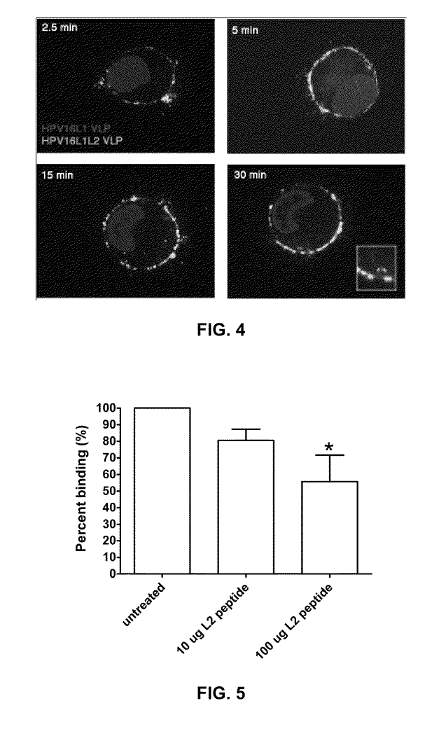

It was determined whether HPV16L1 VLP and HPV16L1L2 VLP enter LC through similar or different cellular compartments. To assess HPV uptake, each VLP was labeled with different fluorescent dyes and incubated LC with both VLP simultaneously. The uptake of the VLP in LC was then visualized by confocal microscopy at various time points. It was shown that while both HPV16L1 VLP and HPV16L1L2 VLP co-localize at the LC surface, they enter and travel through the LC cytoplasm in different compartments as demonstrated by the clear separation of fluorescent dye labeled particles (FIG. 4). This finding indicates that a specific receptor for the L2 protein may exist on LC.

### The HPV16 L2₁₀₈₋₁₂₀ Peptide Inhibits Binding of HPV16L1L2 VLP to LC

To determine whether the N-terminus L2₁₀₈₋₁₂₀ (aa 108-120, LVEETSFIDAGAP) region facilitated attachment in LC, LC was incubated with increasing concentrations of the L2₁₀₈₋₁₂₀ peptide and subsequently exposed the cells to HPV16L1L2 VLP. The amount of bound HPV16L1L2 VLP on the surface of LC was assessed with flow cytometric analysis. It was determined that increased concentrations of the L2₁₀₈₋₁₂₀ peptide resulted in significantly decreased numbers of HPV16L1L2 VLP bound to the LC surface (FIG. 5), indicating that the N-terminus of L2 facilitates HPV16 binding to LC.

### HPV16 L2₁₀₈₋₁₂₀ Binds to a Specific LC Surface Protein

It was then determined which cell surface protein(s) the L2₁₀₈₋₁₂₀ peptide was binding to on LC and blocking HPV16L1L2 VLP from binding to the cells. LC were either incubated with or without the (6×)His-L2₁₀₈₋₁₂₀ peptide and subsequently exposed to a membrane impermeable cross-linking agent, 3,3′-Dithiobis-(sulfosuccinimidylpropionate) (DTSSP). After cross-linking the L2₁₀₈₋₁₂₀ peptide to the cell surface protein(s) it is closely interacting with, LC were lysed and eluted over 10 fractions. Each fraction was separated by electrophoresis and silver stained. Within elution 5, a distinct band was observed by silver stain just above 39 kDa (FIG. 6a). The band was only present in the eluates that were isolated with the (6×)His-L2₁₀₈₋₁₂₀ peptide. The negative control did not have a corresponding band. The unique band at 39 kDa was excised and analyzed by mass spectrometry protein sequencing at the USC Proteomics Core. The majority of the protein in the band was predicted to be annexin A2 (ANXA2).

ANXA2 is predominantly found on the cell surface as a heterotetramer made up of two ANXA2 molecules and two ANXA2 light chain molecules (Glenney (1986) Proc. Natl. Acad. Sci. USA 83:4258-2462; Waisman (1995) Mol. Cell. Biochem. 149-150:301-322). Through immunoblot analysis, it was found that both ANXA2 and ANXA2 light chain were present in the L2₁₀₈₋₁₂₀ peptide pulldown eluates but not in our negative control eluates (FIG. 3b). Thus, these findings indicate that the ANXA2 heterotetramer is interacting with the L2₁₀₈₋₁₂₀ peptide on the surface of LC.

### Secretory Leukocyte Protease Inhibitor Blocks the Uptake of HPV16L1L2 Virus-Like Particles by LC

Next, this example investigated whether SLPI altered the internalization of HPV16L1L2 VLP by LC. LC were pretreated with increasing concentrations of SLPI and then exposed to carboxyfluorescein diacetate, succinimidyl ester (CFDA-SE) labeled-HPV16L1L2 VLP. Following the incubation, Applicants found that as LC were exposed to increasing concentrations of SLPI, LC internalized decreasing amounts of HPV16L1L2 VLP (FIG. 4a).

It was then determined whether this inhibition of uptake was dependent on the presence of the L2 protein. To do so, LC were pretreated with the optimal SLPI concentrations determined from the previous experiment and subsequently exposed to CFDA-SE labeled-HPV16L1 VLP. Notably, untreated LC and SLPI treated LC internalized similar amounts of HPV16L1 VLP (FIG. 4b), indicating that SLPI did not inhibit HPV16L1 VLP uptake. Taken together, these results indicate that the ANXA2 heterotetramer interacts specifically with the L2 protein and is critically involved with the internalization of HPV16L1L2 VLP by LC.

siRNA Mediated Knockdown of ANXA2 in LC Inhibits HPV16L1L2 VLP Uptake

To confirm the role of the ANXA2/p11 heterotetramer in HPV uptake in LC, the expression of ANXA2 was knocked down in LC using small interfering (si)RNA. Fully differentiated LC was transfected with either no siRNA (untreated), control siRNA that does not knockdown any proteins or two different siRNA sequences targeting ANXA2. As shown, significant reduction was achieved in the expression of ANXA2 in LC treated with ANXA2 siRNA compared to both untreated and control siRNA treated LC (FIG. 5a). It was determined that optimal protein knockdown occurred 6 days post-transfection using ANXA2 siRNA #2, which was utilized throughout subsequent experiments. To determine the effect of ANXA2 knockdown on HPV16L1L2 VLP uptake in LC, LC were treated with siRNA and exposed to CFDA-SE labeled-HPV16L1L2 VLP. Specific knockdown of ANXA2 in LC significantly reduced the uptake of HPV16L1L2 VLP into LC compared to both untreated LC and control siRNA treated LC (FIG. 5b). Collectively, these results indicate that the ANXA2 heterotetramer interacts with the L2 protein and is critically involved in the internalization of HPV16L1L2 VLP by LC.

The concept of viruses binding to a single receptor and subsequently entering cells through a single uptake mechanism has been challenged (Marsh and Helenius (2006) Cell 124: 729-740; Mercer et al. (2010) Annu. Rev. Biochem. Available: http://www.ncbi.nlm.nih.gov/pubmed/20196649. Accessed 7 Mar. 2010; Sieczkarski and Whittaker (2005) Curr. Top. Microbiol. Immunol 285:1-23). Instead, a more complex picture is forming where specific co-receptors and multiple attachment sites lead eventually to viral entry by one or multiple uptake mechanisms. The highly evolutionarily conserved amino acid region of L2 108-120 has been shown to be vital in the binding and infectivity of HPV in many cell types (Kawana et al. (2001) J. Virol. 75:2331-6), but until now it has never been shown to be critical in the binding and infectivity of LC. This example demonstrates through binding and pulldown assays that HPV16 L2₁₀₈₋₁₂₀ is critical in the binding of HPV16 VLP to LC and specifically interacts with the ANXA2 heterotetramer on the surface of LC. Through uptake assays, it was shows that the internalization of HPV16L1L2 VLP by LC is mediated by the ANXA2 heterotetramer. The uptake of HPV16 VLP can be inhibited by either SLPI, a known ligand of ANXA2, or siRNA mediated knockdown of ANXA2 in LC. Collectively, these data indicate that the ANXA2 heterotetramer is an uptake receptor for HPV16 on LC.

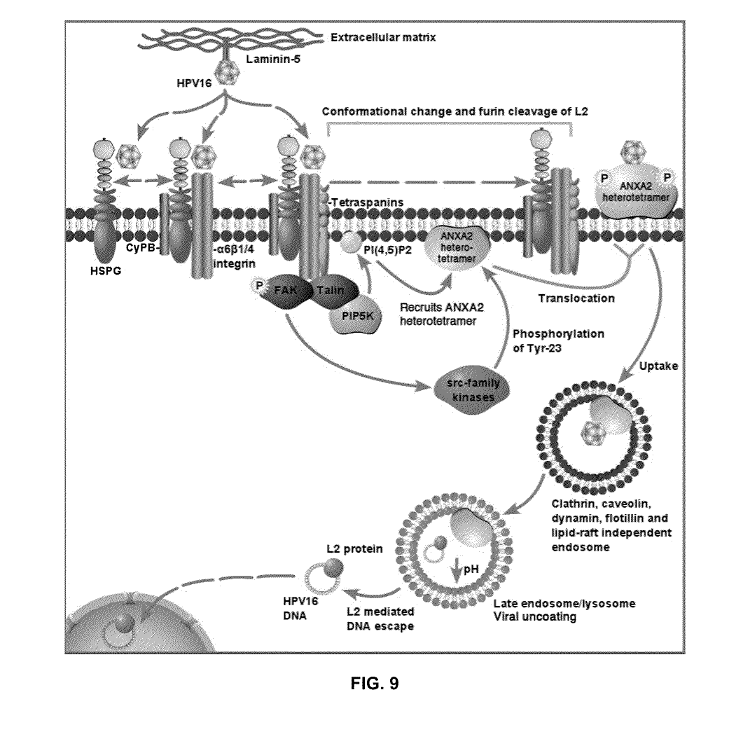

The results in this example indicate that the ANXA2 heterotetramer is an uptake receptor for HPV on LC. It is demonstrated through a variety of methods that the ANXA2 heterotetramer mediates the internalization of HPV16 and that this internalization is dependent on the presence of the L2 protein. Accordingly, the applicant proposes model of HPV uptake that unifies a seemingly disparate and fractured field of research (FIG. 9). In this model, HPV capsids bind to the extracellular matrix (ECM) protein laminin-5 before binding to the host cell surface, although this interaction may not be critical to productive infection. On the host cell surface, HPV initially interacts with HSPG. There are also several other potential cell surface receptors/binders for HPV, including α₆β_{1/4} integrin, CyPB and tetraspanins (CD63 and CD151). All of these cell surface molecules interact with each other and facilitate functionality, such as signaling, and therefore binding of HPV may occur via a singular molecule or a complex of molecules, as shown in FIG. 6. While the functional significance of HPV binding to some of these surface receptors is not fully elucidated, it is clear that interaction of HPV with HSPG results in a conformational change that results in the exposure of a furin cleavage site. This leads to proteolytic cleavage of the L2 protein, resulting in additional conformational changes that decreases the affinity of the capsid for the primary receptor and exposes a binding site for the secondary cell surface receptor. This results in a hand off of the HPV capsid to the secondary receptor, which leads to uptake of HPV through endocytosis. This examples also demonstrates that SLPI, a ligand of the ANXA2 heterotetramer, can inhibit the internalization of HPV.

This example is the first to identify the ANXA2 heterotetramer as the HPV16 L2 receptor on LC that is responsible for the internalization of HPV. This result is surprising because, until now, neither a specific receptor for the HPV L2 protein nor an uptake receptor for HPV has been identified. Furthermore, these data have broad implications because this ANXA2 heterotetramer mediated viral uptake pathway may represent a currently unknown type of receptor-mediated endocytosis. Finally, this example identifies the ANXA2 heterotetramer as a potential therapeutic target for the inhibition of HPV and therefore these findings have future clinical implications on HPV therapy.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for inhibiting human papillomavirus (HPV) infection of a Langerhans cell (LC) or a tissue containing a LC, comprising administering to the LC or the tissue an effective amount of an agent that inhibits HPV binding to annexin A2 (ANXA2) present on the surface of the cell, thereby inhibiting HPV infection.

2. A method for treating or preventing HPV infection in a subject having or at risk of HPV infection, comprising administering to the subject an effective amount of an agent that inhibits HPV binding to ANXA2, thereby preventing or treating HPV infection in the subject.

3. A method for preventing or inhibiting HPV-related pathologies in a subject having or at risk of an HPV infection, comprising administering to the subject an effective amount of an agent that inhibits HPV binding to ANXA2, thereby preventing or treating HPV-related pathologies in the subject.

4. The method of any of claims 1 to 3, further comprising administering an effective amount of a TLR agonist.

5. The method of any of claims 1 to 3, wherein the agent that inhibits binding of HPV to ANXA2 is one or more of a secretory leukocyte protease inhibitor (SLPI), an agent that enhances expression of SLPI, a peptide comprising the extracellular domain of an ANXA2 receptor, an siRNA directed at ANXA2 or p11, a polynucleotide encoding the siRNA, a peptide comprising an HPVL2 region or an antibody that recognizes and binds ANXA2.

6. The method of claim 5, wherein the siRNA comprises (SEQ ID NOS 6 & 8).

7. The method of any one of claims 1 to 3, wherein the HPV binds to ANXA2 by binding to either or both of ANXA2 and/or p11 in the ANXA2/p11 heterotetramer complex.

8. The method of any of claims 1 to 3, further comprising administering an effective amount of a contraceptive.

9. The method of any of claims 1 to 3, wherein the administration is topically.

10. The method of any of claims 1 to 3, wherein the administration is vaginal, rectal, penile, oral, or on skin surface.

11. The method of claim 4, wherein the agent and the TLR agonist are administered concomitantly or sequentially.

12. The method of claim 2 or 3, wherein the subject is a human patient.

13. A method to determine if a test agent is suitable for one or more of: inhibiting or preventing HPV infection of a LC or a tissue containing a LC, or as an antiviral or anticancer agent for cells or tissue that harbor HPV, the method comprising
(a) administering to a first tissue sample an amount of the test agent;
(b) administering to a second tissue sample an effective amount of an agent that inhibits binding of HPV to ANXA2; and
(c) comparing the binding, uptake, or gene expression of HPV in the first tissue sample to the binding, uptake, or gene expression in the second tissue sample and/or third tissue sample, wherein the test agent is determined as suitable as one or more of: an antiviral, as an anticancer agent or for inhibiting or preventing HPV infection of a LC or a tissue containing a LC if the HPV viral titer of the first tissue sample is similar to the second tissue sample.
